(19) United States
(12) Patent Application Publication
(10) Pub. No.: US 2022/0372695 A1
(43) Pub. Date: Nov. 24, 2022

(54) ACTIVE AGENT INDICATOR FOR TEXTILES

(71) Applicant: Textile-Based Delivery, Inc., Conover, NC (US)

(72) Inventors: Michael Drzewinski, Denver, NC (US); Jordan Schindler, Charlotte, NC (US)

(73) Assignee: Textile-Based Delivery, Inc., Conover, NC (US)

(21) Appl. No.: 17/748,630

(22) Filed: May 19, 2022

Related U.S. Application Data
(60) Provisional application No. 63/191,143, filed on May 20, 2021.
(60) Provisional application No. 63/317,784, filed on Mar. 8, 2022.

(51) Int. Cl. D06M 16/00; A41D 31/04; A41D 27/08

(52) U.S. Cl. CPC D06M 16/00; A41D 31/04; A41D 27/08; D10B 2403/02421; D10B 2501/00

(57) ABSTRACT

The present invention relates to textile garments and articles including a functional indicator, and more specifically to textile garments and articles having an indicator that presents a visual change as a function of number of washes and/or active compound concentration. The present invention also discloses systems and methods for using the textile garments and articles including the functional indicator.

### CROSS REFERENCES TO RELATED APPLICATIONS

This application is related to and claims priority from the following U.S. patents and patent applications. This application claims the benefit of U.S. Provisional Application No. 63/191,143, filed May 20, 2021, and U.S. Provisional Application No. 63/317,784, filed Mar. 8, 2022, each of which is incorporated herein by reference in its entirety.

### BACKGROUND OF THE INVENTION

### 1. Field of the Invention

The present invention relates to textile garments and articles including a functional indicator, and more specifically to textile garments and articles having an indicator that presents a visual change as a function of number of washes and/or active compound concentration.

### 2. Description of the Prior Art

It is generally known in the prior art to provide indicators for when garments and articles should be replaced due to wear.

Exemplary prior art references include the following:

WIPO Publication No. WO2020201600 for Marker for objects that indicates the degree of release into the environment of elements that are difficult or slow to biodegrade (harmful elements) by inventor Costa Botey, filed Mar. 30, 2020 and published Oct. 8, 2020, is directed to a marker for objects that indicates the degree of release into the environment of elements that are difficult or slow to biodegrade (harmful elements), which consists of an element having a structure that ostensibly modifies its appearance at a rate in accordance with the wear of the fabric of a garment for which it is intended, alerting the user. The element is a button or a tag comprising two different looking layers, a superficial layer and a central or bottom layer, the external layer being “wearable” and disappearing at a rate in accordance with the fabric of the garment. The publication discloses that the element is stitching consisting of a “wearable” thread that disappears at a rate in accordance with the deterioration of the fabric of the garment for which it is intended.

U.S. Pat. No. 9,578,908 for Apparel with wear indicator by inventors Berns, et al., filed Mar. 28, 2013 and issued Feb. 28, 2017, is directed to a sports bra configured to be worn by a human female includes a body fabric and a wear indicator. The wear indicator is configured to change colors when the useful life of the sports bra has expired and the sports bra should be replaced with a new sports bra. The wear indicator may be provided on any of various components of the sports bra, such as a color band, trim feature, tag, or other sports bra component.

U.S. Pat. No. 11,011,080 for Arrangement for indicating an intended end of the period of use of a textile product by inventor Engl, filed Apr. 15, 2016 and issued May 18, 2021, is directed to an arrangement for indicating an intended end of a period of use of a textile product. The textile product or a label of the textile product has an indicator field which, in a first area, has a first color that changes during the period of use of the textile product and in the course of the associated washes. The indicator field additionally has a second color in a second area, wherein the wash-fastness of the first color is less than the wash-fastness of the second color, and the first color differs, in the delivery state, from the second color and, over a number of washes of the textile product, increasingly approximates to the second color.

U.S. Pat. No. 10,743,604 for Articles of manufacture releasing an active ingredient by inventors Theberge, et al., filed Nov. 29, 2017 and issued Aug. 18, 2020, is directed to articles of manufacture having a formulation distributed therein, wherein the formulation comprises an active agent which manifests a desirable property when released from the articles of manufacture. The patent also relates to methods for manufacturing these articles of manufacture, to vehicles for applying the formulation to these articles of manufacture, and to perception indicators indicating the presence and the amount of formulation comprising an active agent applied to an article of manufacture.

U.S. Pat. No. 10,378,875 for Performance gauge for fabric and cushioning material by inventors Cranin, et al., filed Nov. 7, 2017 and issued Aug. 13, 2019, is directed to athletic apparel, including compression garments and athletic footwear, capable of monitoring the wear of compression fabric and/or cushioning material by attaching a performance gauge onto the apparel. The performance gauge includes microcapsules that contain a colorless dye and/or a co-reactant, wherein the microcapsules can breakdown in concert with the wear and degradation of the compression fabric or cushioning material, thereby allowing the dye and co-reactant to mix and produce color indication. As the wear of the fabric and cushioning material increases, more microcapsules breakdown resulting in a progression of color change visible through additional layers on the performance gauge, or by the fabric and/or cushioning material. The microcapsules can be engineered to breakdown based on any variation of factors that correspond to the degradation of compressive fabric and cushioning material, including shear force, tension, impact force, and/or exposure to high temperature and water.

U.S. Patent Publication No. 20100058973 for Control and/or monitoring method for the salting-out of at least one active ingredient impregnated in a textile by inventor Beauge-Duguet, filed Nov. 15, 2007 and published Mar. 11, 2010, is directed to a control and/or monitoring method for the salting-out of at least one active ingredient impregnated in a first textile material characterized in that: a) said first textile material impregnated with said at least one active ingredient is provided, b) a control tag is affixed on the first textile material, and is made from a second textile material at least partially impregnated with a dye, the quantity of which being adjusted in such a way that the discoloration of said tag is proportional to the salting-out of said at least one active ingredient, c) the quantity of the at least one salted-out active ingredient is evaluated by visualizing the discoloration of the said tag. The publication relates to a control and/or monitoring tag of the salting-out of at least one active ingredient impregnated into a textile material.

U.S. Patent Publication No. 20040038848 for Biocidal cloth by inventor Kritzler, filed May 10, 2001 and published Feb. 26, 2004, is directed to a cleaning article including an agent or reagent, for example a biocidal agent or reagent, which is slowly released therefrom in use of the article and a visual indicator selected to present a change in appearance indicative of the amount of agent or reagent remaining in, or released from, the article. The publication discloses that the cleaning article comprises a nonwoven fabric or sponge containing one or more biocides which are slowly released during use, and bearing a crosslinked ink which fades during use at a rate which indicates when the article is no longer reliably biocidal. The publication also relates to a visual indicator for use on a cleaning article containing one or more biocides which are slowly released during use, said indicator consisting of an ink formulation which is crosslinked to a degree selected so that the ink will wear off or fade to a predetermined degree with the amount of usage that causes the biocide concentration to fall below an effective level. The amount of biocidal agent in or on a cleaning device when the agent is depleted or inactivated during use may be determined by comparing the appearance of the used cleaning device with the appearance of a corresponding unused cleaning device or a colour reference.

U.S. Pat. No. 5,293,648 for Tag for visually indicating loss of a protective agent by inventor Finley, filed Oct. 28, 1991 and issued Mar. 15, 1994, is directed to an indicator tag is provided for visually indicating the remaining effectiveness of a protective chemical treatment agent on a textile article. The tag is made from a fabric dyed with at least one dye to impart to the piece of fabric a predetermined initial color which indicates that the chemical treatment agent on the textile article is effective. The dye has a chromophore which is susceptible to degradation by reagents which would destroy the effectiveness of the chemical treatment agent so that exposure of the indicator tag to such reagents causes the indicator tag to change from its predetermined initial color to another color, thereby indicating that the textile article with which the indicator tag is used has been exposed to reagents which may cause a loss in the effectiveness of the protective chemical treatment agent on the textile article.

U.S. Pat. No. 4,678,704 for Impregnated substrate incorporating an indicator dye by inventor Fellows, filed Jul. 24, 1986 and issued Jul. 7, 1987, is directed to an impregnated fabric material comprising a fabric substrate to which has been bonded an active cationic impregnant characterized in that there has also been applied to the substrate an anionic indicator dye in combination with a further cationic component, and in that the dye bonds to the further cationic component more readily than to the substrate and that the further cationic component competes with the impregnant for bonding to the dye. In the case of a wiping cloth, when the dye, which can act as an indicator, has been removed to indicate exhaustion of the active component, enough active component in fact remains on the cloth to provide a safety margin.

U.S. Pat. No. 4,311,479 for Method of indicating the presence of an impregnant in a substrate by inventors Fenn, et al., filed Jun. 23, 1980 and issued Jan. 19, 1982, is directed to a method of indicating the presence of an impregnant, e.g., an antimicrobial composition, in cloth to provide visual evidence of the continuing activity of the impregnant. The antimicrobial composition in the cloth is activated on contact with a liquid such as water and is ionically bonded to the cloth. Small portions of the impregnated cloth are dyed with an indicator dye which bonds preferentially to the antimicrobial composition so that when the antimicrobial composition is exhausted, the dye will disappear from the cloth. The dye is preferably applied to the cloth in stripes.

U.S. Pat. No. 6,971,935 for Flotation swimsuit and method for construction thereof by inventor Gilmer, filed Nov. 7, 2000 and issued Dec. 6, 2005, is directed to a flotation swimsuit for providing strategically distributed buoyancy to a wearer. The flotation swimsuit includes a form-fitting torso covering which includes a retaining pocket for enclosing a unitary flotation element. The unitary flotation element includes a backsheet formed from a layer of buoyant material having a thickness within a first predetermined range for providing general buoyancy to a wearer and having an inner surface for presentation toward the wearer and an outer surface for presentation away from the wearer. The unitary flotation element also includes a plurality of enhanced buoyancy regions integrally carried by the backsheet for providing enhanced buoyancy to specific areas of the wearer's body. Each of the enhanced buoyancy regions is formed form a buoyant material having a thickness substantially greater than that of the backsheet and each enhanced buoyancy region extends outwardly from the outer surface of the backsheet.

### SUMMARY OF THE INVENTION

The present invention relates to textile garments and articles including a functional indicator, and more specifically to textile garments and articles having an indicator that presents a visual change as a function of number of washes and/or active compound concentration.

It is an object of this invention to provide textile garments and articles containing at least one active compound applied to or incorporated in the yarns, yarn precursors, threads, filaments, and/or fibers thereof including a functional indicator.

In one embodiment, the present invention provides a wearable article including a yarn, a fiber, or a section of fabric, at least one active composition applied to the yarn, the fiber, or the section of fabric, and an indicator, wherein the yarn, the fiber, or the section of fabric releases the at least one active composition when in contact with a skin surface of a wearer, and wherein the indicator provides a visual change that corresponds to an amount of the at least one active composition in the yarn, the fiber, or the section of fabric.

In another embodiment, the present invention provides a wearable article with an indicator including a yarn, a fiber, or a section of fabric including visual indicia that correspond to an amount of at least one active composition applied to the yarn, the fiber, or the section of fabric, wherein the yarn, the fiber, or the section of fabric releases the at least one active composition when in contact with a skin surface of a wearer, wherein the visual indicia undergo a visual change that corresponds to the amount of the at least one active composition present in the yarn, the fiber, or the section of fabric after one or more uses or wash cycles of the yarn, the fiber, or the section of fabric, and wherein the visual change results from a chemical reaction.

In yet another embodiment, the present invention provides a wearable article including a yarn, a fiber, or a section of fabric having a first color associated therewith, at least one active composition applied to the yarn, the fiber, or the section of fabric, and an indicator associated with, chemically bonded to, or complexed with the at least one active composition, wherein the yarn, the fiber, or the section of fabric releases the at least one active composition when in contact with a skin surface of a wearer, wherein the indicator has a second color associated therewith, and wherein an amount of the at least one active composition is correlated with the second color of the indicator.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 depicts a garment with an indicator as disclosed and described herein.

FIG. 2 depicts an exemplary qualitative, semi-qualitative, quantitative, or semi-quantitative wash cycle indicator as disclosed and described herein.

FIG. 3 depicts an exemplary qualitative, semi-qualitative, quantitative, or semi-quantitative wash cycle correlation grid as disclosed and described herein.

FIG. 4 illustrates another embodiment of an indicator in an initial, intermediate, and final state.

FIG. 5A illustrates an active agent indicator system including a plurality of yarns in an initial state.

FIG. 5B illustrates an active agent indicator system including the plurality of yarns from FIG. 5A in a final state.

FIG. 6A illustrates an active agent indicator of weft knit fabric in an initial state.

FIG. 6B illustrates the active agent indicator from FIG. 6A of weft knit fabric in a final state.

FIG. 7A illustrates an active agent indicator of warp knit fabric in an initial state.

FIG. 7B illustrates the active agent indicator from FIG. 7A of warp knit fabric in a final state.

FIG. 8A illustrates an active agent indicator of woven fabric in an initial state.

FIG. 8B illustrates the active agent indicator from FIG. 8A of woven fabric in a final state.

FIG. 9A illustrates an active agent indicator system in an initial state.

FIG. 9B shows the indicator system from FIG. 9A in an intermediate state.

FIG. 9C shows the indicator system from FIG. 9A in a final state.

FIG. 10A illustrates an alternate active agent indicator system in an initial state.

FIG. 10B illustrates the alternate active agent indicator system from FIG. 10A in a final state.

FIG. 11 illustrates a cross-sectional view of FIG. 10A

FIG. 12 illustrates a cross-sectional view of FIG. 10B.

FIG. 13 depicts regression of ΔE₀₀ verses wash cycles for an exemplary dye system with single dye indicator as disclosed and described herein.

FIG. 14 depicts regression of ΔE₀₀ verses wash cycles for an exemplary dye system with dual dye indicators as disclosed and described herein.

FIG. 15 depicts regression of ΔE₀₀ verses wash cycles for and exemplary dye system with multiple dye indicators as disclosed and described herein.

FIG. 16 depicts regression of ΔE₀₀ verses wash cycles for another exemplary dye system with multiple dye indicators as disclosed and described herein.

FIG. 17 depicts an exemplary dye system indicator production process as disclosed and described herein.

FIG. 18 is a schematic diagram of a system of the present invention.

### DETAILED DESCRIPTION

The present invention is generally directed to an indicator for textile garments and articles, and more specifically to an indicator that presents a visual change as a function of number of washes for a textile. In one embodiment, the visual change correlates to a remaining quantity of an active ingredient (e.g., for future delivery) and/or continued proper function of the textile garments and articles.

In one embodiment, the present invention provides a wearable article including a yarn, a fiber, or a section of fabric, at least one active composition applied to the yarn, the fiber, or the section of fabric, and an indicator, wherein the yarn, the fiber, or the section of fabric releases the at least one active composition when in contact with a skin surface of a wearer, and wherein the indicator provides a visual change that corresponds to an amount of the at least one active composition in the yarn, the fiber, or the section of fabric. In one embodiment, the indicator undergoes a chemical reaction to produce the visual change. In one embodiment, the wearable article has an initial state, an intermediate state, and a final state, wherein the initial state does not provide a contrasting indication, wherein the intermediate state provides the contrasting indication, wherein the final state provides a maximum contrasting indication, and wherein the maximum contrasting indication provides a constant contrasting indication. In one embodiment, the visual change includes a quick response (QR) code, wherein, upon being scanned by a camera or sensor of an electronic device, the QR code provides a link to a website or opens an application to facilitate purchase of the wearable article. In one embodiment, the indicator is a colorimetric indicator. In one embodiment, the indicator is a quinone or a para-substituted phenolic compound. In one embodiment, the at least one active composition has a first release rate from the yarn, the fiber, or the section of fabric when in contact with the skin surface of the wearer, wherein the indicator has a second release rate from the yarn, the fiber, or the section of fabric, and wherein the second release rate is substantially similar to the first release rate to correlate with an amount of the at least one active composition of the yarn, the fiber, or the section of fabric. In one embodiment, the indicator is associated with, chemically bonded to, or complexed with the at least one active composition. In one embodiment, the at least one active composition includes a capsaicinoid. In one embodiment, the wearable article is an ankle sleeve, an arm sleeve, a calf sleeve, a knee sleeve, a lower leg sleeve, a wrist sleeve, a wrap, a sock, an insole, a glove, tights, leggings, partial leggings, pants, partial pants, a shirt, or a partial shirt.

In another embodiment, the present invention provides a wearable article with an indicator including a yarn, a fiber, or a section of fabric including visual indicia that correspond to an amount of at least one active composition applied to the yarn, the fiber, or the section of fabric, wherein the yarn, the fiber, or the section of fabric releases the at least one active composition when in contact with a skin surface of a wearer, wherein the visual indicia undergo a visual change that corresponds to the amount of the at least one active composition present in the yarn, the fiber, or the section of fabric after one or more uses or wash cycles of the yarn, the fiber, or the section of fabric, and wherein the visual change results from a chemical reaction. In one embodiment, the chemical reaction produces or reduces a visual presence of a color, a symbol, and/or text characters. In one embodiment, the chemical reaction includes the at least one active composition and one or more reactants. In one embodiment, the chemical reaction includes the one or more reactants and a second reactant. In one embodiment, the one or more reactants are coupled to or immobilized on an inorganic support or an organic support. In one embodiment, the chemical reaction is reversible. In one embodiment, the chemical reaction is an acid-base reaction, a decomposition reaction, or an oxidation-reduction reaction. In one embodiment, the one or more reactants includes a quinone or a para-substituted phenolic compound. In one embodiment, the one or more reactants includes Gibbs reagent.

In yet another embodiment, the present invention provides a wearable article including a yarn, a fiber, or a section of fabric having a first color associated therewith, at least one active composition applied to the yarn, the fiber, or the section of fabric, and an indicator associated with, chemically bonded to, or complexed with the at least one active composition, wherein the yarn, the fiber, or the section of fabric releases the at least one active composition when in contact with a skin surface of a wearer, wherein the indicator has a second color associated therewith, and wherein an amount of the at least one active composition is correlated with the second color of the indicator.

Washing lifespan is useful for fabrics and garments. In one example, washing lifespan is useful for fabrics and garments including at least one active compound applied to or incorporated in the yarn, yarn precursor, thread, filament, fiber, textile, or fabric. Medicated yarns, yarn precursors, threads, filaments, and/or fibers are woven or knitted into garments, such as compression wear, socks, and leggings, or articles, such as pillowcases. The medicated yarns, yarn precursors, threads, filaments, and/or fibers include, for example, at least one active compound configured to administer to the wearer or user during use (e.g., for pain relief). The at least one active compound content in the garment or textile decreases during use as well as after laundering, i.e., wash cycles. Examples of fabrics and garments containing at least one active compound applied to or incorporated in the yarns, yarn precursors, threads, filaments, fibers, textiles, or fabrics thereof are disclosed in U.S. Pat. Nos. 9,669,012 and 10,799,464, U.S. Patent Publication Nos. 20210363687, 20200390720, 20190038568, and 20170231919, U.S. patent application Ser. No. 17/678,612, and U.S. Provisional Patent Application No. 63/191,143, each of which is incorporated herein by reference in its entirety.

Prior art documents generally disclose indicators for when garments should be replaced due to wear. However, none of the prior art discloses an indication of the number of wash cycles for garments and articles incorporating medicated yarns, yarn precursors, threads, filaments, fibers, textiles, or fabrics.

In one example, a textile wash cycle indicator system is provided, the indicator system including an initial amount of a dye system that includes at least one dye, the dye system configured to release an amount of the at least one dye during consecutive wash cycles. In one embodiment, the release of the amount of the at least one dye correlates with the consecutive wash cycles.

In another example, the release of the at least one dye correlates with a visual change of the dye system. In a preferred embodiment, the release of the at least one dye correlates to a visual change of the dye system; an availability of the at least one active compound in the garments and articles including the at least one active compound applied to or incorporated in the yarns, yarn precursors, threads, filaments, fibers, textiles, or fabrics thereof; a releasability of the at least one active compound applied to or incorporated in the yarns, yarn precursors, threads, filaments, fibers, textiles, or fabrics thereof; and/or a retention of at least one coating applied to or incorporated in the yarns, yarn precursors, threads, filaments, fibers, textiles, or fabrics thereof. In another example, alone or in combination with any of the previous examples, the visible change includes, but is not limited to, a change of color; producing, reducing, or increasing a visual presence of a symbol; producing, reducing, or increasing a visual presence of text characters; or combinations thereof. In another example, alone or in combination with any of the previous examples, the visible change is irreversible.

In another example, the present invention provides an indicator of functionality (e.g., via release of the at least one dye). In one embodiment, the functionality includes, but is not limited to, wear (e.g., loss of stretch); lifespan of the garment or article; environmental pollution; presence of at least one active compound; an availability of the at least one active compound in the garments and articles including the at least one active compound applied to or incorporated in the yarns, yarn precursors, threads, filaments, fibers, textiles, or fabrics thereof; a releasability of the at least one active compound applied to or incorporated in the yarns, yarn precursors, threads, filaments, fibers, textiles, or fabrics thereof; a retention of at least one coating applied to or incorporated in the yarns, yarn precursors, threads, filaments, fibers, textiles, or fabrics thereof; corrections on use or treatment of the garment or article; and/or next steps regarding use of the garment or article (e.g., reorder, lifespan ended, etc.).

In one example, the indicator provides a visual indication corresponding to or correlating with a qualitative or quantitative amount of the at least one active compound present and/or remaining in the garment or article, analogous to a “fuel gauge.” The indicator is operable to be integral with or coupled to the textile garment. For example, and not limitation, in one embodiment, the indicator is a part of a care label, a logo associated with the textile garment or article, or an inconspicuous section of the textile garment or article. In one embodiment, the indicator is manufactured separately and apart from the textile garment or article and assembled, coupled, or associated with the garment or article during production.

In another example, alone or in combination with any of the previous examples, the dye system is integral with a textile. In another example, alone or in combination with any of the previous examples, the dye system is integral with a yarn, yarn precursor, thread, filament, fiber, textile, or fabric of a garment or article. In one example, the garment or article further includes at least one yarn, yarn precursor, thread, filament, fiber, textile, or fabric including at least one active compound.

In another example, alone or in combination with any of the previous examples, the dye system includes: a first dye having a first washing fastness value; and one or more dyes different from the first dye, each of the one or more dyes different from the first dye independently having a washing fastness value less than the first wash fastness value. In another example, alone or in combination with any of the previous examples, the rate of change of at least one of the washing fastness values less than the first wash fastness value corresponds to a first range of wash cycles.

In another example, alone or in combination with any of the previous examples, the rate of change of at least one of the washing fastness values less than the first wash fastness value corresponds to a second range of wash cycles greater than or less than the first range of wash cycles. In another example, alone or in combination with any of the previous examples, the rate of change of at least one of the washing fastness values less than the first wash fastness value corresponds to a range of wash cycles between 1 and about 10 and where the rate of change of at least one of the washing fastness values less than the first wash fastness value corresponds to a range of wash cycles of more than about 10.

In another example, alone or in combination with any of the previous examples, the rate of change of at least one of the washing fastness values less than the first wash fastness value corresponds to a range of wash cycles between 1 and about 10 and where the rate of change of at least one of the washing fastness values less than the first wash fastness value corresponds to a range of wash cycles of between about 10 to about 20 and where the rate of change of at least one of the washing fastness values less than the first wash fastness value corresponds to a range of wash cycles greater than 20.

In another example, alone or in combination with any of the previous examples, the first dye or the one or more dyes different from the first dye are coupled to or immobilized on an inorganic support or an organic support. In another example, alone or in combination with any of the previous examples, the first dye and the one or more dyes different from the first dye are coupled to or immobilized on an inorganic support or an organic support.

In one embodiment, the indicator includes a substance having strong color characteristics that is altered at a predictable, reproducible rate when the textile garment or article is in use (e.g., by a user) or after repeated wash/dry cycles of laundering. For example, and not limitation, the indicator includes a reactant that undergoes a chemical reaction with the therapeutic active or reactant chosen to correlate with the therapeutic active. The contrasting result of this reaction is either the generation of a visible color, a visible color change, or the loss or fading of a visible color. In one example, the indicator includes a reactant that undergoes a reaction with a second reagent, resulting in generation of a visible color, a visible color change, or the loss or fading of a visible color. In one example, the second reagent is similar to the therapeutic agent present in the textile such that the reaction with the contrasting indicator is operable to be indirectly correlated with that of the therapeutic active. By similar, it is meant that the diffusion properties, molecular weight, polymeric matrix, reactivity, and/or other physical and chemical properties provide for the indirect correlation with that of the therapeutic active. For example, and not limitation, a color-indicating agent is coupled with capsicum oleoresin and the like for purposes of providing the indicator for a textile that includes cannabidiol.

In one embodiment, the term “substantially” refers to a tolerance level of a term modified by the term “substantially.” For example, a first release rate of a therapeutic active from a textile fiber is at least “substantially” the same as a second release rate of a color-indicating agent if the first release rate is within ten percent (10%) of the second release rate. In one example, this tolerance is 9%, 8%, 7%, 6%, 5%, 4%, 3%, 2%, 1%, or less than 1%.

Another example includes a method for indicating wash cycles of a textile garment or article. The method also includes providing an indicator associated with a yarn, yarn precursor, thread, filament, fiber, fabric, or section of fabric of the textile garment or article that directly or indirectly corresponds to a qualitative, semi-qualitative, quantitative, or semi-quantitative amount of wash cycles of a textile. The method also includes providing a visual change of the indicator that directly or indirectly corresponds to the qualitative or the quantitative amount of washing cycles of the textile. The method also includes providing an indicator associated with a yarn, yarn precursor, thread, filament, fiber, fabric, or section of fabric of the textile garment or article that directly or indirectly corresponds to a qualitative, semi-qualitative, quantitative, or semi-quantitative amount of wash cycles of a textile; an availability of the at least one active compound in the garment or article including the at least one active compound applied to or incorporated in the yarns, yarn precursors, threads, filaments, fibers, textiles, or fabrics thereof; a releasability of the at least one active compound applied to or incorporated in the yarns, yarn precursors, threads, filaments, fibers, textiles, or fabrics thereof and/or a retention of at least one coating applied to or incorporated in the yarns, yarn precursors, threads, filaments, fibers, textiles, or fabrics thereof.

Referring now to the drawings in general, the illustrations are for the purpose of describing one or more preferred embodiments of the invention and are not intended to limit the invention thereto.

Embodiments of this disclosure provide yarns, yarn precursors, threads, filaments, fibers, fabrics, and other textiles, and other substrates that release therapeutically effective amounts of active compounds (e.g., organic active compounds) to the skin of a mammal. Such active compounds are operable to be selected for their dermatological and/or cosmetic benefit, e.g., for skin health and beauty. The active compounds are operable to penetrate into the skin or be delivered to tissue below the skin, including to the bloodstream. In certain embodiments, the active compound(s) are operable to penetrate into or through the skin to a depth that depends on the active concentration, the yarn-to-skin (or substrate-to-skin) contact time, physicochemical properties of the active, and/or the structure and condition of the skin.

Embodiments of this disclosure also provide yarns, yarn precursors, threads, filaments, fibers, textiles, and/or other substrates that release a therapeutically effective amount of active compound into the bloodstream of a mammal from outside the body. For instance, in one embodiment, this includes transdermal delivery, wherein contact of the yarn, the yarn precursor, the thread, the filament, the fiber, the textile, or the substrate with mammalian skin results in transfer of one or more active compounds through the skin and into the bloodstream. Textiles, fabrics, articles, clothing, or apparel including yarns, yarn precursors, threads, filaments, fibers, and/or other substrates that deliver or release therapeutic amounts of active compounds to, or through, the skin of a mammal that makes contact with the textile, the fabric, the article, the clothing, or the apparel are also provided.

Embodiments of this disclosure also provide yarns, yarn precursors, threads, filaments, fibers, textiles, fabrics, and/or substrates that are able to withstand washing and other stresses (e.g., physical, chemical, thermal, weather) with minimal or no loss of active. Thus, the present invention provides cold washable and hot-washable yarns, yarn precursors, threads, filaments, fibers, textiles, fabrics, and/or substrates that are loaded with active. For example, in a normal washing machine hot wash cycle, these yarns, yarn precursors, threads, filaments, fibers, textiles, fabrics, and/or substrates are operable to lose less than about 25%, less than about 12%, less than about 7%, less than about 3%, or less than about 1% of the active that was present in the material just before the wash.

The present disclosure also includes active agent indicators that are able to withstand washing and other stresses (e.g., physical, chemical, thermal, weather) and maintain correlation with the active agent present and/or remaining in the textile. Thus cold washable and hot washable active indicators are provided. For example, in a normal washing machine hot wash cycle, the active indicator is configured to correlate with active-releasing fabrics or yarns that are designed to lose less than about 25%, less than about 12%, less than about 7%, less than about 3%, or less than about 1% of the active that was present in the material just before a first wash or use or a subsequent wash or use.

in one embodiment, at least one substrate (e.g., yarn, yarn precursor, thread, filament, fiber) is used to form a fabric or a textile. In certain embodiments, the fabric or the textile formed with the yarn, yarn precursor, thread, filament, or fiber includes both the medicated yarn of this disclosure along with an ordinary, non-medicated yarn, yarn precursor, thread, filament, or fiber. For example, in woven textiles, the warp is operable to be traditional yarn and the weft is operable to be yarn of the present embodiments. In other embodiments, only medicated yarn, yarn precursor, thread, filament, or fiber is used. In one example, the fabric or textile includes one or more antimicrobial yarns in a knitted or woven configuration with one or more yarns including a releasing therapeutic active (e.g., copper- or silver-infused or coated yarn in a knitted or woven configuration with yarn including a releasable therapeutic active).

In one embodiment, the textile formed with the yarn or filament is a knitted textile. In one embodiment, the knitted textile is a circular knit. Alternatively, the knitted textile is a flat knit. In one embodiment, the knitting of the textile is facilitated by treating the yarns of the present disclosure with a lubricant (e.g., 2% to 3% lubricant) prior to knitting.

Advantageously, the knitted textile is operable to provide both a relaxed state and a stretched state. In one embodiment, the wearable article or garment is formed of the knitted textile. In a preferred embodiment, the wearable article or garment is operable to substantially conform to at least one body part of a wearer. The at least one body part includes, but is not limited to, a finger, a hand, a wrist, an elbow, an arm, a shoulder, a torso, a back, a head, a face, a neck, a toe, a foot, an ankle, a knee, a hip, and/or a leg. In one embodiment, the wearable article or garment produces a compressive force when worn. In one embodiment, the wearable article or garment is operable to provide compression to the at least one body part of the wearer. In one embodiment, the compression is between about 5 mmHg and about 60 mmHg. In another embodiment, the compression is between about 15 mmHg and about 50 mmHg. In yet another embodiment, the compression is between about 20 mmHg and about 40 mmHg. In one embodiment, the compression is between about 5 mmHg and about 15 mmHg, about 15 mmHg and about 20 mmHg, about 20 mmHg and about 30 mmHg, about 30 mmHg and about 40 mmHg, or about 40 mmHg and about 50 mmHg. In one embodiment, the wearable article or garment is seamless. Advantageously, a seamless wearable article or garment provides additional comfort to a wearer by reducing points of friction.

In some embodiments, the substrate is a yarn, yarn precursor, thread, filament, fiber, textile, or fabric. As used herein, the terms “yarn” and “yarn precursor” include not only finished yarns, but also starting or intermediate fiber-based materials from, e.g., greige cotton or extruded filament, to finished—and, as described in certain embodiments, functionalized yarns (e.g., yarns that are loaded with an active compound), e.g., whether on a cone or spool or in a textile or fabric. The term “yarn” is also operable to describe individual threads and spun and/or twisted threads. In one embodiment, the yarn is bulked or textured. In one embodiment, bulked and/or textured yarns refer to yarns treated mechanically, chemically, and/or physically (e.g., tension-adjusted) to appear to have greater or increased volume relative to the yarn prior to mechanical, chemical, and/or physical treatment. For example, but not limitation, bulked and/or textured yarns are operable to have a crimped, coiled, or spiral configuration rather than a linear or stretched configuration. Bulked and/or textured yarns are operable to exhibit favorable properties over, e.g., partially-oriented yarn (POY) or other yarns lacking texture and/or bulk. In certain embodiments, the yarn includes a nylon, polyester, or acrylic material.

In one embodiment, the wearable article forms an orthopedic cast, splint material, a wound dressing, socks, hats, face/ski masks, scarves, tiaras, chokers, skullcaps, undergarments, skin guards, wrist bands, arm bands, knee pads, bras, shirts, leggings, tights, nylon stockings, athletic supporters, protective athletic equipment (e.g., gloves, pads, and helmets), robes, neck bands, head bands, ear muffs, gloves, diapers, poultices, facial masks, paraffin gloves, and/or joint braces. In one embodiment, the present invention provides a wearable article including, but not limited to, an ankle sleeve, an arm sleeve, a calf sleeve, a knee sleeve, a lower leg sleeve, a wrist sleeve, a shirt or a partial shirt, pants or partial pants, leggings or partial leggings (e.g., ⅞ leggings, capri, shorts), an insole, a sock, or a glove. In one embodiment, the wearable article is a brace including, but not limited to, an ankle brace, an arm brace, a knee brace, a lower leg brace, a wrist brace, a finger brace, a shoulder brace, a neck brace, a back brace, or a hip brace. In one embodiment, the wearable article is a splint including, but not limited to, an ankle splint, an arm splint, a knee splint, a lower leg splint, a wrist splint, a finger splint, a shoulder splint, a neck splint, a back splint, or a hip splint.

In one embodiment, the wearable article is unitarily formed from the textile, the fabric, or the substrate. In one embodiment, the wearable article is operable to be pulled onto the at least one body part. For example, but not limitation, a knee sleeve is operable to be pulled over the foot and the calf, and placed over the knee. In another example, a shirt is operable to be pulled over the head, and arms of the wearer pulled through sleeves of the shirt.

In still another embodiment, the wearable article is a wrap. In one embodiment, the wrap is operable to be wrapped around the at least one body part. For example, but not limitation, the wearable article is a back brace operable to secure around a torso of a wearer. In another example, the wearable article is an elastic bandage (e.g., ACE bandage) operable to wrap around the at least one body part (e.g., arm, leg).

In another embodiment, the textile, the fabric, or the substrate forms a layer in the wearable article. For example, and not limitation, in one embodiment, the textile, the fabric, or the substrate forms a first layer of the wearable article in contact with surface of the skin (e.g., a lining) and a second layer of the wearable article is exposed to the environment.

In certain embodiments, the yarn or substrate requires greater elasticity or stretch. Thus, in one embodiment, the yarn is plied or twisted with an air-covered yarn (e.g., spandex) to enable additional stretch of the yarn. Additionally, the yarn is operable to be air-covered/air-intermingled (i.e., blowing air onto the yarn and adding a spandex core into the middle of the yarn). These methods are particularly useful for garments that need a lot of stretch such as tights, leggings, or an elastic portion on a top of a sock or an ankle sleeve. In one embodiment, the wearable article is operable to stretch to conform to the at least one body part of a wearer.

In one embodiment, the wearable article further includes at least one closure mechanism. The at least one closure mechanism includes, but is not limited to, at least one strap, at least one snap, hook and loop tape, at least one tie, at least one buckle, at least one zipper, at least one lace, at least one closure system (e.g., BOA fit system), at least one latch, at least one hook, at least one elastic, at least one adhesive, at least one fastener, and/or at least one clip. In one embodiment, the at least one closure mechanism allows for customization of fit of the wearable article to the wearer. In another embodiment, the at least one closure mechanism secures the wearable article to at least one body part of the wearer. For example, but not limitation, hook tape and loop tape secure a wrist brace to a wrist of a wearer. In another example, the at least one closure mechanism is a tie to secure pants to a waist of a wearer. In still another example, at least one hook or at least one clip secures an elastic bandage (e.g., ACE bandage) to the at least one body part (e.g., arm, leg).

In one embodiment, the present invention provides an article (e.g., non-wearable article) that is operable to release at least one active composition. In one embodiment, the article is formed using yarns, yarn precursors, threads, filaments, fibers, textiles, and/or substrates (e.g., films, sheets, cut and sew pieces) that are operable to persistently release the at least one active composition. Advantageously, the article and/or the yarns, yarn precursors, threads, filaments, fibers, textiles, and/or substrates are operable to persistently release the at least one active composition after at least one wash cycle. In one embodiment, the yarns, yarn precursors, threads, filaments, fibers, textiles, and/or substrates form a knitted structure. Advantageously, the knitted structure is operable to provide both a relaxed state and a stretched state. In one embodiment, the article is formed of the knitted structure. Alternatively, the yarns, yarn precursors, threads, filaments, fibers, textiles, and/or substrates form a woven structure. In one embodiment, the yarns, yarn precursors, threads, filaments, fibers, textiles, and/or substrates are operable to be used for cut and sew. The article is operable to contact at least one body part of a wearer. In one embodiment, the article is operable to substantially conform to the at least one body part of a user. The at least one body part includes, but is not limited to, a finger, a hand, a wrist, an elbow, an arm, a shoulder, a torso, a back, a head, a face, a neck, a toe, a foot, an ankle, a knee, a hip, and/or a leg. In one embodiment, the article is seamless. Advantageously, a seamless article provides additional comfort to a user by reducing points of friction. In an alternative embodiment, the article or garment is formed of a woven textile.

In one embodiment, the article includes a towel, a cover, a pillowcase, a sheet (e.g., flat sheet, fitted sheet), a comforter, a blanket, upholstery, drapes, a mat (e.g., yoga mat, exercise mat, anti-fatigue mat), a grip (e.g., athletic equipment, handlebars), and/or a sleeping bag or sleeping bag liner. In one embodiment, the cover includes a seat cover, an electric device cover (e.g., mouse cover, keyboard cover, phone cover), a heating pad cover, a duvet cover, ice pack cover, steering wheel cover, a furniture cover, and/or an exercise apparatus cover (e.g., bench cover). In one embodiment, the article is an upholstered article.

In one embodiment, the article further includes at least one closure mechanism. The at least one closure mechanism includes, but is not limited to, at least one strap, at least one snap, hook and loop tape, at least one tie, at least one buckle, at least one zipper, at least one lace, at least one closure system (e.g., BOA fit system), at least one latch, at least one hook, at least one elastic, at least one adhesive, at least one fastener, and/or at least one clip. In one embodiment, the at least one closure mechanism allows for customization of fit of the article to a platform (e.g., seat, steering wheel) and/or another article (e.g., pillow, duvet, mattress). In one embodiment, the article is operable to create contact with skin of a user via user to surface pressure. For example, but not limitation, at least one elastic is used to secure a fitted sheet to a mattress. In another example, at least one strap is used to secure a seat cover to a seat. In still another example, user to surface pressure is used to contact the article (e.g., seat pad) to the skin of the user.

A structural integrity of the wearable article and/or the article is preferably maintained after at least one wash. In a preferred embodiment, a size and/or a shape of the wearable article and/or the article does not substantially change after at least one wash. The wearable article and/or the article is preferably reusable as the wearable article and/or the article, respectively, after at least one wash. Prior art articles including superabsorbent polymers are not washable, and do not maintain their structural integrity after washing. For example, a disposable diaper including superabsorbent polymers cannot be reused as a diaper following at least one wash. The size and/or the shape of the diaper is substantially changed after at least one wash.

Certain embodiments also provide athletic garments and undergarments and other sportswear/active wear releasing one or more of the following: performance-enhancing actives; aspirin, local anesthetic, and/or capsaicin or a capsaicinoid for relief of pain or cold; creatine, glutamine, citrulline malate, beta-alanine, and/or branched-chain amino acids for muscle recovery or muscle stimulation; and handkerchiefs releasing cologne or perfume, antimicrobials, and/or vitamins.

In certain embodiments, solid active particles or powders (e.g., crystalline active particles) are used. Solid active particles (e.g., crystalline active particles) are operable to be used, in part, to better achieve dissolution-limited release kinetics. Exemplary forms of active compounds that are operable to be used include, but are not limited to, crystalline or polycrystalline solid particles, semi-crystalline solid particles, amorphous solid particles, plant extracts including crystalline or amorphous solid domains of one or more active compounds from the plant, and mixtures or combinations thereof. In further embodiments, the active compounds are operable to include components or fractions of plant essential oils. In one embodiment, the components or factions of plant essential oils are crystalline at room temperature and suitable for use. The term “plant essential oils” is as described in U.S. Patent Application Publication No. 2014/0271863, which is incorporated herein by reference in its entirety, and which also provides a listing of some of the organic compounds that provide for the desirable or therapeutic effects of these oils.

In some embodiments, the active includes a heating or cooling active. For example, in one embodiment, the active is selected from at least one of menthol, a menthol derivative, WS compounds (Wilkinson Sword™) (e.g., WS-3, 5, 12, and 23), methyl salicylate, ethyl salicylate, trolamine salicylate, capsaicin or a capsaicinoid, isopulegol, N-ethyl-2-isopropyl-5-methylcyclohexanecarboxamide (WSE-500), a synthetic heating or cooling agent (e.g., nonivamide), vanillyl butyl ether, ginger, eugenol, kunzea, arnica (e.g., arnica oil), camphor, niacinamide, and/or diphenyl hydramine. In certain embodiments, the active includes a cooling component blend (e.g., isopulegol, menthyl lactate, menthoxypropanediol, and 2-isopropyl-N,2,3-trimethylbutyramide (WS-23)). Other suitable heating or cooling actives are also within the scope of this disclosure.

In various embodiments, the active includes an anti-fungal active. For example, in one embodiment, the active is selected from at least one of clotrimazole, miconazole, ketoconazole, terbinafine, fluconazole, and/or amphotericin. Other suitable anti-fungal actives are also within the scope of this disclosure.

In some embodiments, the active includes an antipruritic and/or skin calming active. For example, in one embodiment, the active is selected from at least one of an antihistamine (e.g., diphenhydramine or hydroxyzine), a corticosteroid (e.g., hydrocortisone), a counterirritant (e.g., mint oil, menthol, or camphor), and/or a local anesthetic (e.g., lidocaine, pramoxine, benzocaine, trolamine, calamine, coenzyme Q-10, and/or diphenyl hydramine). In one embodiment, the active includes a mixture of lidocaine and prilocaine (e.g., EMLA). Other suitable antipruritic and/or skin calming actives are also within the scope of this disclosure.

In certain embodiments, the active includes an acne-treating active. For example, in one embodiment, the active is selected from at least one of an alpha-hydroxy acid (AHA) (e.g., glycolic acid or lactic acid), benzoyl peroxide, clay, salicylic acid, sulfur, tea-tree oil, azelaic acid, topical retinoid, and/or kojic acid. Other suitable acne-treating actives are also within the scope of this disclosure.

In various embodiments, the active includes an emollient. In one embodiment, the emollient is selected from at least one of shea butter, cocoa butter, a castor oil derivative, lanolin, squalene, coconut, jojoba, sesame, almond, another plant oil and/or butter, cetyl alcohol and derivatives, and/or a stearate. Other suitable emollients are also within the scope of this disclosure.

Other suitable actives include, but are not limited to, cannabidiol, BEAUPLEX® VH; ALL-Q® (coenzyme Q10) plus; coenzyme, SPECIKARE™ CQ10; ROVISOME™ Q10; SIGNALINE™ S, SERENITYL™ BIOFUNCTIONAL; ATPEPTIDE™ IS; PROLIPID™ 141; VITAL ET™; GENTI-FOL® SA; CELLULINO®; lidocaine; oleoresins (e.g., capsicum oleoresin); SHAPEPERFECTION™; FRESH'N™ CC menthol 50% (CYCLOSYSTEM COMPLEX®); CARNIPURE™ CRYSTALLINE; SYNIORAGE™ LS 9847; ULTRA FILLING SPHERES™; RONACARE® nicotinamide; VEXEL™ SP; cafeisilane C; caffeine (e.g., ROVISOME™ caffeine10; ISOCELL™ SLIM; DISILANOL C+™; SYNIORAGE™ LS 9847, N,N-Diethyl-meta-toluamide (DEET), picaridin, and/or melatonin. Other suitable actives are also within the scope of this disclosure (see, e.g., Table 1).

TABLE 1

| | | Skin Energizing/ | |
|---|---|---|---|
| Skin Sensations | Skin Calming | Renewal | Skin Firming |
| Heat | Acne | Hydration | Anti-wrinkles |
| Cool | Irritation | Cellular Energy | Cellulite |
| Odor, Itch | Rosacea/Psoriasis | Protection | Anti-Glycation |
| Capsaicin | Emollients | Vitamins A, C and E | Carnosine |
| Nonivamide | Lidocaine | Glycolic Acid+ | Carnitine |
| Menthol | CoQ-10 | Resveratrol | Vitamin Bs, E & C |
| WS Derivatives | Cinnamates | Polyphenols | Peptides |
| Miconazole | Piroctone Olamine | Niacinamide | Salicylates |
| Diphenhydramine | Calamine | Glycyrrhizinic Acid+ | Alpha hydroxyl acids |
| | | Ergothioneine | |
| | | Ferulic Acid | |
| | | Caffeine | |

In one embodiment, a concentration of the active composition or agent is in a range from about 250 ppm to about 100,000 ppm. The specific concentration is selected based on the pharmacological attributes of the active agent(s) employed, the polymer, the yarn or fiber, the application method, any post-polymerization treatments and/or finishing steps applied to the textile, and the like. In one embodiment, the indicator is configured to correlate with the concentration of the active agent present in the textile garment or article and to reflect changes in the concentration of the active agent during normal use and repeated wash cycles. In one example, the indicator includes the same active agent present in the textile garment or article. In another example, the indicator includes a reagent different from the active agent present in the textile garment or article.

In one embodiment, the indicator includes a diffusible matrix, the diffusible matrix including a reagent, an active or reactant physically separated from the reagent within the diffusible matrix, or the reaction product of the active or the reactant with the reagent. In one embodiment, the diffusible matrix is configured to diffuse the active or the reactant, or the reaction product of the active or the reactant with the reagent from the matrix at a predetermined rate of diffusion. In one embodiment, alone or in combination with any one of the previous embodiments, the predetermined rate of diffusion is non-zero first order.

For example, in one example, the polymer matrix includes an elastomer in which the active, reactant, and/or reagent are dispersed. In one embodiment, the reagent is immobilized on a support or on particles. In one example, the support or particles are silica or silica nanoparticles.

Exemplary elastomers include, but are not limited to, silicones, rubbers, halogenated rubbers, polyether block amides, ethylene vinyl acetates, elastolefins, polyurethane elastomers, fluoropolymer elastomers (fluoroelastomers), thermoplastic elastomers (TPEs), and mixtures and combinations thereof. In one embodiment, the fluoropolymer elastomers are operable to repel hydrocarbons. The polymer matrix is also operable to include an elastomer blended or otherwise mixed with other polymers. In one embodiment, the elastomer domains are continuous from one nd protect them from end of the elastomer domain to another end such that the active, reactant, or reagent is operable to diffuse in and/or out of the elastomeric domains. For example, an illustrative polymer matrix includes both elastomeric domains and crystalline domains, where the elastomeric domains are continuous.

In one embodiment, cross-linking of the polymer matrix is performed so as to lock, hold, or otherwise temporarily retain the active, reactant, or reagent in place and protect them from degradation and premature loss, particularly in the face of stress conditions (e.g., those encountered in laundering). In one embodiment, the polymer matrix is cross-linked in the presence of dispersed or suspended active, reactant, or reagent, which are coated along the longitudinal axis or the length of the yarn or substrate (e.g., a section of fabric).

Indicator Systems

As previously described, the present disclosure provides an indicator that is operable to indicate qualitatively or quantitatively a predetermined number of wash cycles for the fabric, garment, or article. In one example, the predetermined number of wash cycles is at least 1, 5, 10, 15, 20, 25, or more wash cycles. In another example, the predetermined number of wash cycles is between about 3 and about 25. In another example, the predetermined number of wash cycles is between about 2 and about 50. In one example, a perceivable indicia change is operable to be observed after each subsequent wash throughout 5, 10, 15, or 25 or more wash cycles. In a preferred embodiment, the indicator provides a sensitivity of±5 washes for long duration use (e.g., at least 25 wash cycles).

The present disclosure provides a wash cycle indicator system suitable for a fabric, garment, or article, the indicator configured to alter its visual appears in qualitative, semi-qualitative, quantitative, or semi-quantitative relationship to the number of wash cycles of the fabric, garment, or article. In one example, the indicator is configured for reflecting approximately 15, approximately 20, and/or approximately 25 washes or more of the fabric, garment, or article. In another example a single dye system is constructed for providing visual indication of approximately 15, approximately 20, and/or approximately 25 washes or more of the fabric, garment, or article.

In one aspect of the present disclosure, the system includes one or more dyes associated with a yarn, yarn precursor, thread, filament, fiber, textile, or section of a fabric, garment, or article. The one or more dyes associated with a yarn, yarn precursor, thread, filament, fiber, textile, or section of a fabric, garment, or article are configured such that during or after each successive wash, a small amount of the one or more dyes fade out. After a predetermined number of washes, the one or more dyes are operable to completely fade out. In another aspect of the present disclosure, the system is configured such that during or after each successive wash, a small amount of the one or more dye is operable to fade out. In one embodiment, the one or more dyes fade out after 5 washes, 10 washes, 15 washes, 20 washes, or 25 washes, etc. The one or more dyes are operable to completely fade out at a predetermined wash cycle limit. For example, and not limitation, the yarn, yarn precursor, thread, filament, fiber, textile, or section of a fabric, garment, or article starts out with an initial visual indication and after a predetermined number of washes, presents a visual indication different from the initial visual indication.

In one embodiment, the one or more dyes are included in a coating. In one embodiment, the coating is lost via washing such that the one or more dyes are lost over time as the coating washes out. Advantageously, incorporating the one or more dyes into a matrix (e.g., coating) in which the one or more dyes is lost with the matrix over time prevents dye leaching.

In one embodiment, the one or more dyes include a first dye and a second dye. In one embodiment, the first dye and the second dye have different responses to wash. For example, and not limitation, the first dye is a red dye and the second dye is a blue dye, wherein the blue dye washes out faster than the red dye. The first dye and the second dye are combined to produce a purple color, which fades to red with loss of the blue dye.

In one embodiment, the indicator is a colorimetric indicator. In one embodiment, the colorimetric indicator includes, but is not limited to, ninhydrin, fluorescamine, bromophenol, chloranil, Ellman's reagent (5,5′-dithiobis-(2-nitrobenzoic acid), dansyl hydrazine, Brady's reagent (2,4-dinitrophenylhydrazine), PURPALD reagent (e.g., 4-amino-3-hydrazino-5-mercapto-1,2,4-triazole), Gibbs reagent (2,6-dichlorobenzoquinone-4-chloroimine), pyridine and iron (III) chloride, ferrous ammonium sulfate, and/or silver nitrate. Ninhydrin is operable to detect primary amines (e.g., diphenhydramine) by reacting with the deprotected N-terminal amine group to produce an intense blue color. Fluorescamine is a spiro compound that is not fluorescent itself and reacts with primary amines (e.g., bacitracin) to form highly fluorescent products. Bromophenol is operable to detect primary and/or secondary amines (e.g., pseudoephedrine) and produces a deep blue color in the presence of free amino groups and a greenish yellow color in the absence of free amino groups. Chloranil is operable to detect a secondary amine (e.g., phenylephrine) by producing a brown/red/orange derivative by displacing chloride from the ring of the quinone. Ellman's reagent is operable to cleave a disulfide bond in a thiol (e.g., captopril) to produce TNB ions (2-nitro-5-thiobenzoate or TNB⁻) that are ionized to the TNB dianion (TNB²⁻) in water to give a yellow color. Dansyl hydrazine is operable to detect an aldehyde and/or a ketone (e.g., hydrocortisone) by transfer of the acyl group to the fluorescent hydrazide. Brady's reagent is operable to detect an aldehyde and/or a ketone (e.g., camphor) by forming a precipitate between the amine group on the reagent and the carbonyl group. PURPALD reagent is operable to detect an aldehyde (e.g., cinnamaldehyde) by forming an amino acetal that reacts under aeration to form a purple-colored oxidation product. Gibbs reagent is operable to detect a phenol (e.g., acetaminophen) to produce a redox indicator (e.g., phenol reacts with Gibbs reagent to produce 2,6-dichlorophenolindophenol). Pyridine and iron chloride are operable to react with a phenol (e.g., oxybenzone) to produce a colored iron complex. Ferrous ammonium sulfate is operable to detect a nitro group (e.g., nitroimidazole) via formation of a red-brown precipitate of iron (III) hydroxide formed by the oxidation of iron (II) hydroxide by the nitro groups. Silver nitrate is operable to detect a halide (e.g., phenoxybenzamine chloride) by reacting with the halide to form silver halide complexes as precipitations. The colorimetric indicator preferably produces a visible color change in the presence of the active compound. Alternatively, the colorimetric indicator is operable to be detected by laboratory equipment (e.g., high-performance liquid chromatography, fluorometer), e.g., for quality assurance purposes.

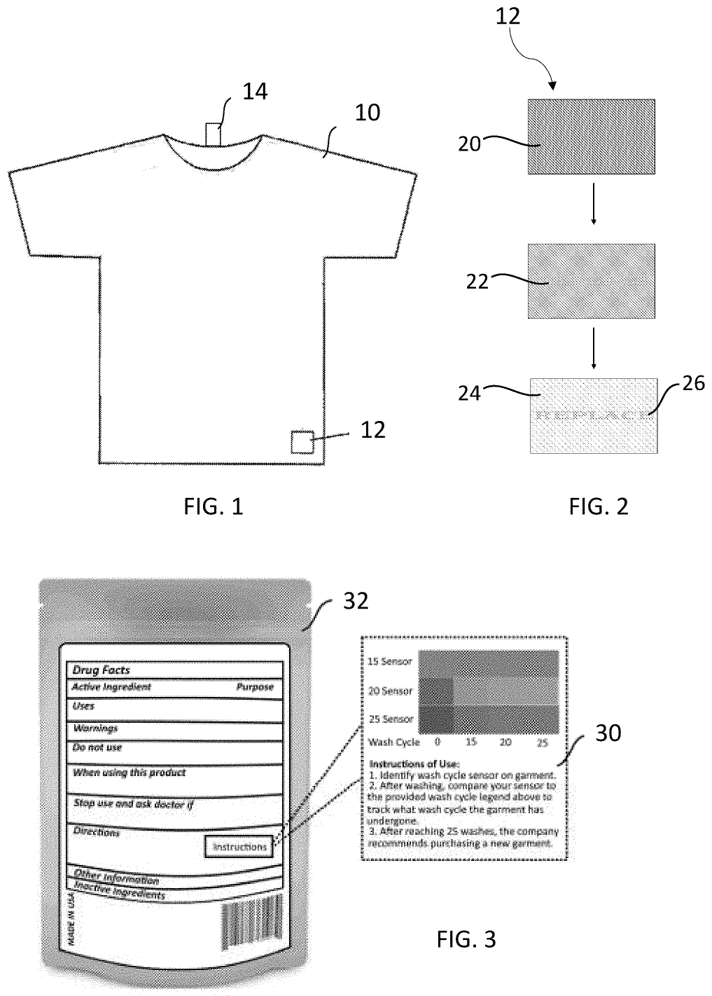

With reference to FIG. 1, an exemplary garment 10 is shown having exemplary indicator 12. In one embodiment, the indicator includes or is part of a care label 14. Alternatively, the indicator is a separate section of garment 10. In one example, indicator 12 is formed of the same fabric as the garment. In another example, indicator 12 is formed of a different fabric than the garment.

Exemplary indicator 12 is shown in FIG. 2, which undergoes a visual transition corresponding to one or more wash cycles of the indicator. In one example, indicator 12 has an initial visual indication 20, which represents the end users purchased product prior to the end user performing a wash cycle on the product. After one or more washes, exemplary indicator 12 transitions to an intermediate visual indication 22. After reaching a predetermined number of wash cycles, the exemplary indicator 12 transitions to a substantially final visual indication 24. In one example, the intermediate or final visual indication presents text or one or more symbols 26. In one embodiment, the text or one or more symbols 26 includes a code (e.g., QR code). The text or one or more symbols 26 are operable to be provided by screen printing the dye(s) or other methods.

In an alternate embodiment, the indicator undergoes a continuous, semi-continuous or stepwise visual transition and the visual transition is referenced on a grid 30 for providing a frame of reference to determine qualitatively or quantitatively the number one cycles experience by the product as shown in FIG. 3. For example, and not limitation, the grid 30 is operable to be provided on the product packaging 32 or available on the manufacturer's web site.

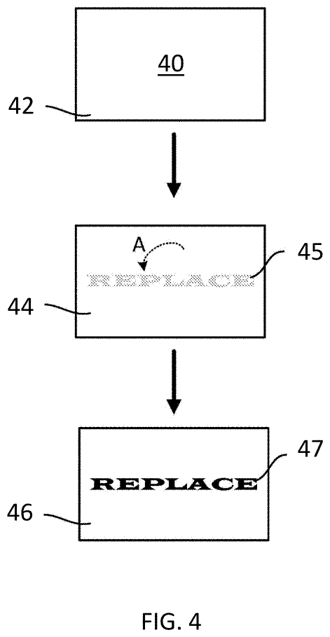

FIG. 4 illustrates another embodiment of an indicator in an initial, intermediate, and final state. A section or tag 40 is configured for attachment to a textile garment or article. The section or tag 40 is shown with substantially no contrasting indication in an initial state 42, such as prior to first use by an end-user of the textile garment or article. The section or tag 40 includes an active or a reactant that chemically reacts with a reagent, resulting in a change that provides for a contrasting indication of the section or tag 40. In one example, the active or the reactant is physically separated from reagent in the section or tag 40, for example, in a matrix. In one embodiment, the matrix is a polymer matrix operable to provide for a specific diffusional rate of the active or reactant. In one embodiment, the reagent is immobilized and substantially inhibited from diffusion within the matrix such that the active or reactant is required to diffuse the reagent. In one embodiment, diffusion of the active or reactant to the immobilized reagent is configured to be the same or substantially similar to the diffusion of the active in the textile garment or article. In one embodiment, the active is the same or one of a plurality of actives present in the textile garment or article. In one embodiment, the reactant is chosen to have similar diffusional properties to that of the one or more of a plurality of actives present in the textile garment or article. In the initial state 42, the section or tag 40 has substantially no contrasting indication. Upon use (e.g., one or more wash cycles) of the section or tag 40 transitions to an intermediate state 44, where a contrasting indication is developed that is visible to the user, such as text or symbols 45. In one embodiment, the text or symbols 45 include a code (e.g., QR code). Upon continued use (e.g., additional wash cycles), the amount of active present in the textile garment or article is substantially or completely exhausted, the section or tag 40 presents in a final state 46, where a maximum level of contrasting indication is achieved, such as with maximally contrasted text or symbols 47. In one embodiment, the maximally contrasted text or symbols 47 indicate that the textile garment or article should be replaced.

In one embodiment, a specific quantitative amount of the remaining active(s) in the textile is not necessary, as the therapeutic active depletion or relative rate of depletion is readily discernible by the wearer (e.g., after washing and drying and/or after using the textile garment or article).

Embodiments disclosed herein are operable to provide an indication (e.g., directly or indirectly) when the therapeutic active is nearing depletion or is depleted in the textile garment or article. As a result, the indicator informs when it is time to buy a new textile product or recharge a depleted textile product. In one embodiment, the textile or garment includes a quantitative or qualitative color-indicating measure of depletion of the therapeutic active. In one embodiment, the indicator includes one or more color-indicating agents (e.g., colorimetric indicator). Color-indicating agents provide several advantages, such as providing the user visual confirmation of activity and aiding in manifesting compliance in the case of a therapeutic treatment regimen.

In one example, a textile includes a first textile fiber including a therapeutic active and additional fibers without the active, and the ratio of the first fiber to that of the additional fibers is selected with regard to the dosing regimen desired. The first textile fiber is configured to release the therapeutic active at a first rate over time when used to administer the therapeutic active to a subject. In one example, the textile also includes a second textile fiber including an active indicator system. In one example, the second textile fiber is configured to cause a contrasting indication corresponding to the depletion of the active from the textile.

In some embodiments, the first textile fiber is used to form a portion or substantially all of the textile, and the second textile fiber forms only a small portion or portions of the textile. By way of non-limiting example, the second textile fiber is used in a logo, a patch, an indicator strip, or other small inconspicuous portion of the textile.

In one example, a textile includes a textile fiber and an active carried by the textile fiber. The therapeutic active has a first release rate from the textile fiber when in use by a subject. The textile also includes an indicator system (e.g., an active indicator system) carried by the same or adjacent textile fiber providing for a contrasting indication corresponding to the relative amount of active. In one example, the contrasting indication is the production or depletion of a color-indicating agent. In one embodiment, the color-indicating agent has correlated to its production or depletion a second release rate from the textile fiber when in use by the subject that is at least substantially the same as the first release rate to correlate with an amount of the active of the textile fiber.

In one example, a textile includes a textile fiber having a first color associated therewith and a therapeutic active carried by the textile fiber. The therapeutic active has a second color associated therewith. An amount of the active carried by the textile fiber is correlated with an effective color of the textile fiber that depends on the first color of the textile fiber, the second color of the therapeutic active, and the amount of the active carried by the textile fiber. The active is configured to release over time from the textile fiber when in use by a subject using the textile.

In one example, a textile includes a textile fiber having a first color associated therewith. The textile also includes an active carried by the textile fiber. The active is configured to release over time from the textile fiber when in use by a subject using the textile. The textile also includes a color-indicating agent that is associated with, chemically bonded (e.g., covalently, ionically, etc.) to, or complexed with the active. The color-indicating agent has a second color associated therewith. An amount of the active carried by the textile fiber is correlated with an effective color of the textile fiber that depends on the first color of the textile fiber, the second color of the color-indicating agent, and the amount of the active carried by the textile fiber.

In one example, the color-indicating agent is selected based on the color-indicating agent having similar or substantially similar physicochemical properties or characteristics to the active. In one example, the color-indicating agent is selected based on the color-indicating agent having similar or substantially similar diffusional properties or characteristics to the active in the polymer matrix used in the active indicator system.

In yet another embodiment, chemical reaction is used as a qualitative indicator of therapeutic agent in a textile, such as an analytical reagent or a bound analytic agent specific for certain chemical functionality that is characteristic of a given therapeutic agent. The qualitative determination of remaining therapeutic present in the textile garment or article is operable to be directly or indirectly correlated with the chemical reaction or chemical reaction equilibrium of the analytical reagent and the therapeutic active. In such examples, the analytical reagent is the equivalent of the color-indicating agent as disclosed and described herein.

In one example, the color-indicating agent is a para-substituted phenolic compound. In another example, the color-indicating agent is a quinone. In yet another example, the color-indicating agent is Gibbs reagent (2,6-dichlorobenzoquinone-4-chloroimine). The Gibbs reagent is rapidly hydrolyzed in basic solution (pH>8), yielding 2,6-dichlorobenzoquinone imine, which reacts at the para position of a phenol ring of certain therapeutic actives such as capsaicin (nonivamide). Additional details about the Gibbs reagent is included in U.S. Provisional Application No. 63/191,143, which is incorporated herein by reference in its entirety.

Optimal conditions for laundering for many commercial detergents is in a liquid that has a pH between 9 and 10.5 (e.g., water that is adjusted to have a pH between 9 and 10.5), and a temperature of about 122° F. (50° C.). Such conditions are typically favorable for use of the Gibbs reagent to qualitatively or quantitatively identify actives that are present in active-containing garments or articles (e.g., capsaicin). In another example, the Gibbs reagent and capsaicin diffusion and color-indicating functionality are operable to be correlated with the change in concentration of other active agents present in the garment by using, for example, linear or polynomial regression.

Most liquid detergents have a pH close to 9, while powdered detergents are usually closer to 10, which are suitable conditions for use of the Gibbs reagent. Thus, in one example, the environment in a washing machine activates the Gibbs reagent and results in reaction with one or more reagents that chemically react with the Gibbs reagent (e.g., capsaicin) release from the yarn coating.

In another example, the Gibbs reagent and an active that chemically reacts with the Gibbs reagent are pre-reacted to provide a contrasting visual indication either in a yarn or a tag or section of the garment or article, which upon multiple washings loses its contrasting indication in a manner that correlates with an amount of the active present in the garment or article. In one example, the Gibbs reagent and a reactant are pre-reacted to provide a color contrasting indication either in yarn or a tag or section of the garment or article, which upon multiple washings loses it contrasting indication in a manner that correlates with an amount of active present in the garment or article, where the reactant is different from the active.

In one example, two yarns (side-by-side) including one with a reagent (e.g., capsaicin, nonivamide) that will chemically react with the Gibbs reagent, and one with the Gibbs reagent without the reagent (e.g., capsaicin, nonivamide), both in physically isolated coatings, are provided. During exposure to perspiration or wash cycle fluids these two coated yarns cause migration of one or both reagents within their respective coatings, resulting in a color contrasting change of the indicator. Alternatively, both the reagent (e.g., capsaicin) and the Gibbs reagent are present in one coating but physically isolated. In both above cases, the yarn is operable to provide a color contrasting indication when wetted by wash water and/or perspiration and is operable to return to a non-color contrasting state when dry.

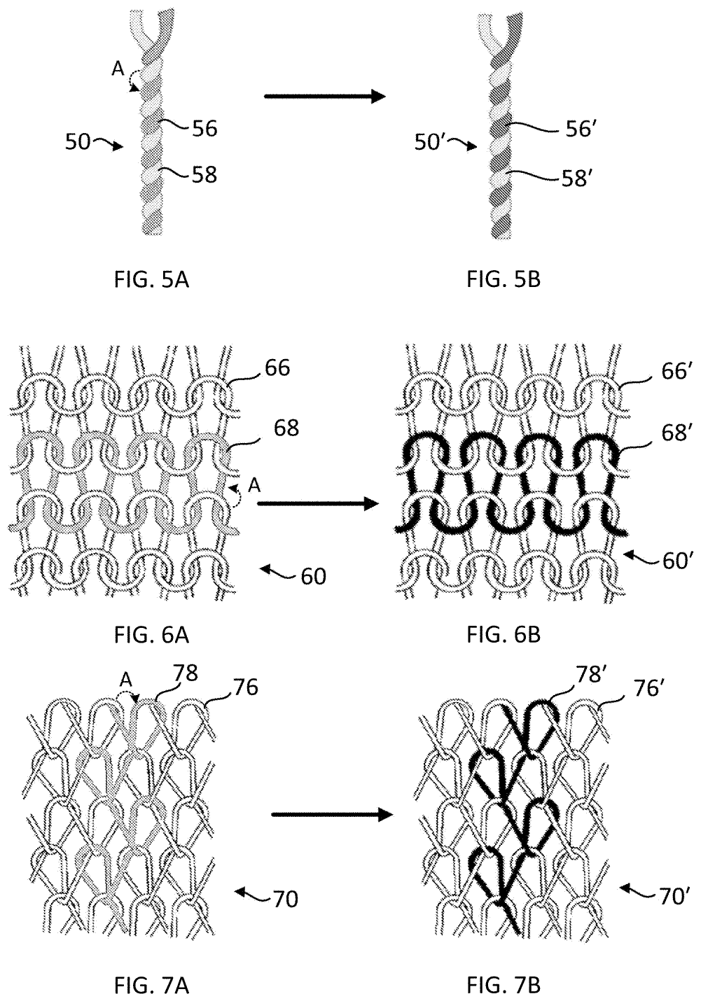

FIG. 5A illustrates an active agent indicator system including a plurality of yarns (e.g., side-by-side). Twisted yarn in an initial state 50 includes a first yarn 56 with a reagent and a second yarn 58 with an agent or reactant. In one example, the reagent of first yarn 56 is immobilized and present in a first matrix. In another example, the active or reactant of second yarn 58 is contained in a second matrix that is the same or different from the first matrix. During use of the garment, as active or reactant diffuses from second yarn 58, some of this active or reactant diffuses into first yarn 56 (shown as arrow “A”) and causes a chemical reaction resulting in a contrasting change (e.g., a color change). In one example, the amount of reagent present in the first yarn 56 is substantially less than stoichiometric equivalent of the total amount of active or reactant present in the second yarn 58. In one example, the stoichiometric ratio of active or reactant to that of the reagent is chosen such that the calculated maximum amount of active or reactant that is operable to be released from the second yarn 58 provides the maximum contrasting indication. In other words, when substantially all of the active or reagent has diffused out of the garment, the contrasting indication remains constant. FIG. 5B illustrates a twisted yarn in a final state 50′ (e.g., the maximum contrasting indication) including the first yarn 56′ and the second yarn 58′.

FIGS. 6A-6B illustrate an alternate active agent indicator of a weft knit fabric in an initial state (FIG. 6A) and a final state (FIG. 6B) functioning in a manner analogous to the indicator system of FIGS. 5A-5B. An alternate active agent indicator of weft knit fabric is shown in an initial state 60 and a final state 60′ functioning in a manner analogous to the indicator system of FIGS. 5A-5B. The embodiment shown in FIG. 6A includes a first yarn 66 with a reagent and a second yarn 68 with an agent or reactant. FIG. 6B illustrates a final state 60′ (e.g., the maximum contrasting indication) including the first yarn 66′ and the second yarn 68′.

FIGS. 7A-7B illustrate an alternate active agent indicator of a warp knit fabric in an initial state (FIG. 7A) and a final state (FIG. 7B) functioning in a manner analogous to the indicator system of FIGS. 5A-5B. An alternate active agent indicator of warp knit fabric is shown in an initial state 70 and a final state 70′, functioning in a manner analogous to the indicator system of FIGS. 5A-5B. The embodiment shown in FIG. 7A includes a first yarn 76 with a reagent and a second yarn 78 with an agent or reactant. FIG. 7B illustrates a final state 70′ (e.g., the maximum contrasting indication) including the first yarn 76′ and the second yarn 78′. Any conventional machine knitting pattern is operable to be used to prepare the indicator system of the present disclosure.

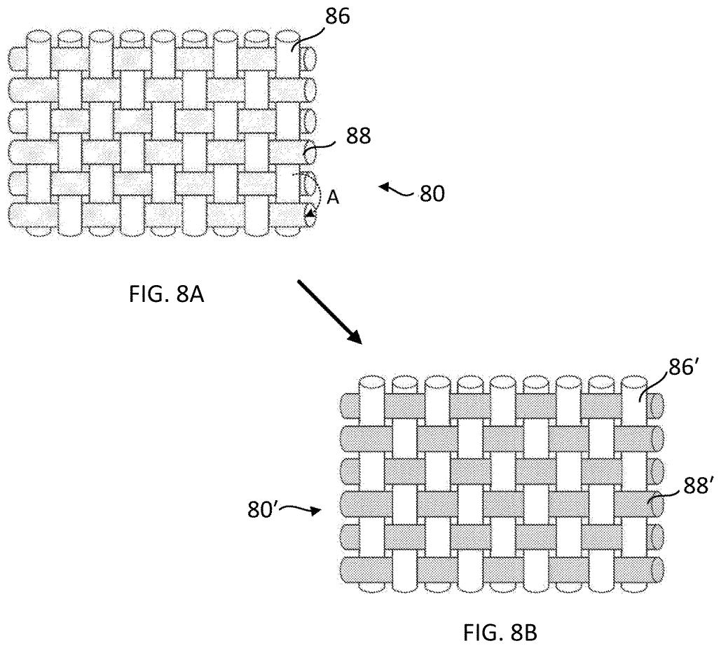

FIGS. 8A-8B illustrate an alternate active agent indicator of woven fabric in an initial state 80 and a final state 80′ functioning in a manner analogous to the indicator system of FIGS. 5A-5B. The embodiment shown in FIG. 8A includes a first yarn 86 with a reagent and a second yarn 88 with an agent or reactant. FIG. 8B illustrates a final state 80′ (e.g., the maximum contrasting indication) including the first yarn 86′ and the second yarn 88′. The first yarn is operable to be the warp and/or the weft. The second yarn is operable to be the warp and/or the weft. Although the embodiment shown in FIG. 8B illustrates the first yarn as the warp and the second yarn as the weft, the first yarn is also operable to be the weft and the second yarn is operable to be the warp. A plain weave, a twill weave, a satin weave, a basket weave, Jacquard weave, Dobby weave, or Leno weave, for example, are operable to be used for preparing the indicator system of the present disclosure.

Alternatively, the initial state is configured with the greatest contrasting indication in the initial state, and upon substantially all of the active or reagent diffusing out of the garment, the final state provides essentially a non-contrasting indication. Thus, in one example, active or reactant are pre-reacted with the reagent to provide a contrasting color either in yarn or a section or tag of the garment or article, which upon multiple washings loses its color-contrasting indication in a manner that correlates with an amount of active present in the garment or article. For example, a contrasting yarn, tag, or section is pre-made that when dry is white or a low contrast, and each time the yarn, tag, or section is washed will increase its contrast until the reactant-containing yarn is depleted.

In one example, the reagent is Gibbs reagent. In another example, the reagent (e.g., Gibbs reagent) is immobilized. In yet another example, the reagent is Gibbs reagent and the active or reactant is capsaicin. In yet another example, the Gibbs reagent is supported on silica and immobilized in a coating diffusional to capsaicin so as to ensure any contrasting—change is specific to the yarn and not lost into the wash water or perspiration.

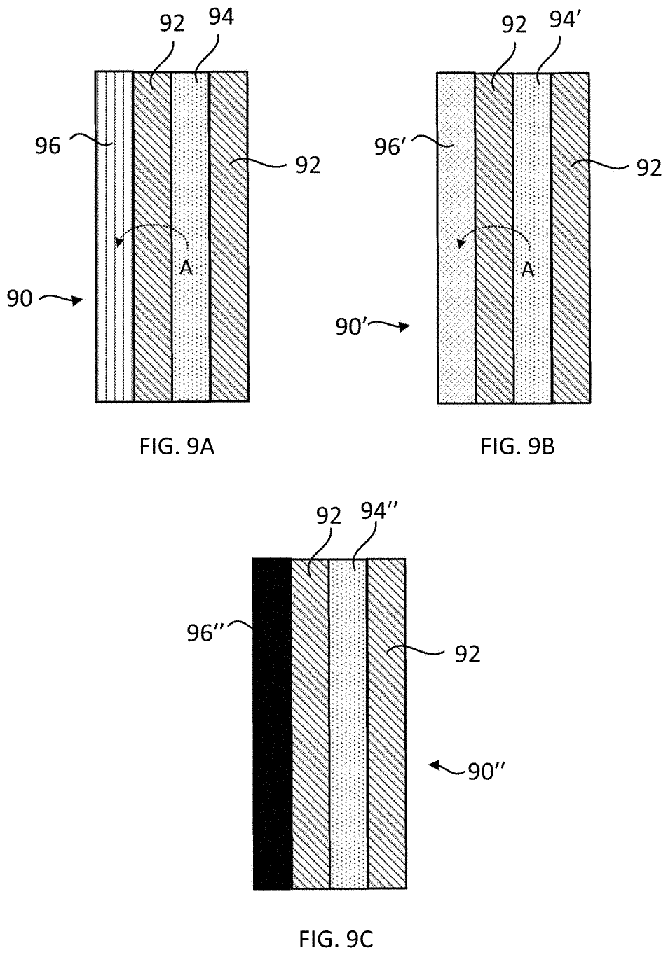

FIGS. 9A-9C illustrate an active agent indicator system in an initial state 90, an intermediate state 90′, and a final state 90″. The indicator system includes a multilayer construct. In an initial state 90, diffusion layers 92 sandwich the agent or reactant layer 94 and physically separate the agent or reactant layer 94 from a reagent layer 96. In one example, the physically separated diffusional layers 92 are the same. In another example, the diffusional layers 92 include different polymer matrices and/or are of different thicknesses so as to modulate and/or control the fusion of agent or reactant from layer 96. In one example, the diffusional layers 92 are configured such that the diffusion of the agent or reactant is substantially or completely unidirectional towards the reagent layer 96. In one example, the reagent layer 96 further includes a plurality of layers, with each layer including a different concentration of reagent. In one example, the reagent layer 96 includes a plurality of layers where the layers in closest proximity to the diffusional layers 92 contain a first concentration of reagent and each successive plurality of layers more distant from the diffusional layers 92 contain concentrations of reagent greater than the first concentration so as to incrementally increase the contrasting indication observed by the user as the active or reactant diffuses from layer 92 into reagent layer 96. Thus, as shown in FIG. 9B, in an intermediate state 90′ the agent or reactant has diffused from the agent or reactant layer 94′ as shown by arrow “A” through the diffusion layer 92 in closest proximity to the reagent layer 96′, thereby causing an intermediate contrasting indication, such as a color change. FIG. 9C shows the indicator system in a final configuration 90″ where substantially all of the agent or reactant has diffused from the agent or reactant layer 94″ into reagent layer 96″, resulting in a final contrast indicating state. Other configurations of the individual layers are compatible with the present invention.

Alternatively, the initial state is configured with the greatest contrasting indication, and upon substantially all of the active or reagent diffusing out of the garment, the final state provides a non-contrasting indication or essentially a non-contrasting indication. In one example, the active or reactant are pre-reacted with the reagent to provide a maximum contrasting color in layer 96″ which upon multiple washings loses it color-contrasting indication in a manner that correlates with an amount of active present in the garment.

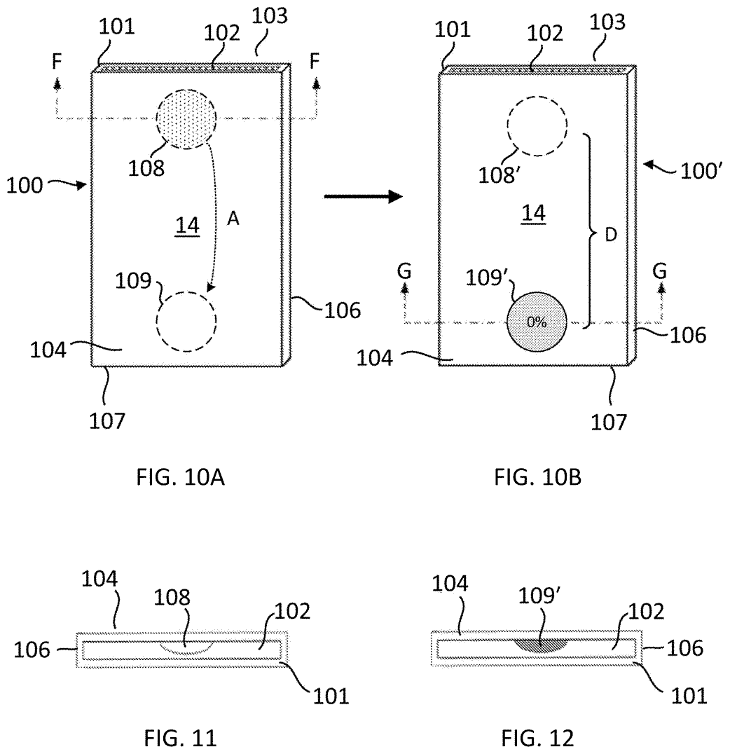

FIGS. 10A-10B illustrate an alternate active agent indicator system in an initial state 100 and a final state 100′. The indicator system includes a physically separated active or reactant and reagent in a 3D construct (e.g., a rectangular shape) having an opening 103 to allow for the introduction and exit of material, with the remainder of the 3D construct having a surface 104, opposing edges 106, and a bottom 107 containing a medium 102. In one example, the surface 104, the opposing edges 106, and the bottom 107 are substantially non-diffusional to the active or reactant but are operable to be diffusional to water or an aqueous medium. In the initial state 100 shown in FIG. 10A, the indicator system has non-contrasting indicating sections 108 and 109 spatially separated with section 108 being in closer proximity to the opening 103 than section 109. In one example, section 108 contains an amount of active or reactant whereas section 109 contains an amount of reagent. During use, the indicator system transitions to an intermediate/final state 100′ as depicted in FIG. 10B, where at least a portion of the active or reactant has diffused from section 108 through the medium 102 as shown by arrow “A” at least in part in the direction of section 109, either by the introduction of water or aqueous medium through opening 103, for example, during wash cycles and/or during application of heat. Some or all of the active or reactant is introduced to section 109 and a contrasting indicating change occurs in section 109 due to the reaction of the active or reactant with the reagent such that a visible change is apparent to the user and quantitatively or qualitatively correlates with the active present in the garment to which the indicator system is attached. In one example, surface 104 of the indicator system is substantially transparent to assist in the visibility of the contrasting indicating change occurring in section 109. Adjusting the distance D between sections 108 and 109 in conjunction with the nature of the medium 102 allows for the correlation of the active present in the garment to be represented by the active indicator system. In one example, medium 102 is the same or similar to the polymer matrix imbibing the active in the construction of the textile garment or article.

FIG. 11 illustrates a section view along sectional line F-F of FIG. 10A showing section 108 as a reservoir of active or reactant. In one embodiment, the reservoir includes a water-soluble polymer (e.g., polyvinyl alcohol). Section 108 is operable to be prepared by screen printing or other deposition techniques for providing a reservoir or pod within a thin-film structure. FIG. 12 depicts a section view along sectional line G-G of FIG. 10B showing a similar reservoir of reagent representing section 109 in an intermediate or final state showing the contrasting indication change as a result of the reaction of the agent or reactant with the reagent. In one example, the reservoir of reagent representing section 109 is configured to preserve and/or protect the reagent from decomposition prior to the introduction of the active or reactant.

In one embodiment, the textile article or garment includes a first indicator and a second indicator. In one embodiment, the first indicator is a wash cycle indicator and the second indicator is an active indicator. For example, and not limitation, the wash cycle indicator correlates to a number of wash cycles and the active indicator correlates to one or more active compounds in the textile garment or article.

Dyes of Dye System

In one example, the dye system includes at least one dye chosen from semi-permanent hair dyes. Semi-permanent hair dyes typically are configured for release from hair over the course of consecutive shampoos. Therefore, semi-permanent hair dyes are operable to be used to correlate to a number of wash cycles. Advantageously, semi-permanent hair dyes are also safe for contact with skin. The dye system of the present disclosure is operable to include one or more dyes selected from the five main classes of dye: acid dyes, direct dyes, disperse dyes, mordant dyes, and/or basic dyes. In another example, the dye system of the present disclosure is operable to include one or more dyes having chemical structures that are of medium molecular weight (less than 1000 Mw) and/or contain at least one acid dye.

In another example, the dye system of the present disclosure includes one or more acid dyes that are negatively charged (anionic) compounds. In one embodiment, the one or more acid dyes includes at least one compound including a sulfonate auxochrome group. In one embodiment, the dye system includes at least one compound that denatures or loses color fastness under alkaline pH.

In yet another example, the dye system of the present disclosure includes one or more basic dyes that are cationic (positively charged) compounds. In another example, the dye system of the present disclosure includes one or more direct dyes that are cationic or anionic, highly soluble in water, and applied to the fibers/yarns in solutions containing ionic salts and electrolytes. In yet another example, the dye system of the present disclosure includes one or more disperse dyes used for dyeing synthetic fibers (e.g., cellulose acetate, polyester, and nylon). In yet another example, the dye system of the present disclosure includes one or more mordant dyes that use mordant for improving the color retention property of the dye molecules within the fiber.

In one example, the dye system includes at least one dye providing a linear rate of change of a visual indicia (e.g., wash fastness) with respect to successive wash cycles. In another example, the dye system includes at least one dye providing a polynomial rate of change with respect to successive wash cycles. In another example, the dye system includes at least one dye providing a linear rate of change of a visual indicia with respect to successive wash cycles in combination with one or more dyes providing a polynomial rate of change with respect to successive wash cycles.

Exemplary Dye Systems

In one example, the dye system is based on a visual indicia, based on or correlated to, a ΔE equation. There are a number of ΔE equations that are operable be used to calculate the difference between two colors or a change of color. In one example, the equation for ΔE₂₀₀₀ (=ΔE₀₀) is used. The numerical value for ΔE₀₀ is operable to be correlated to a human's eye perception of color change approximately 75-80% of the time. Alternatively, the ΔE_{Lab} equation is operable to be used.

Using ΔE values to correlate rate of color change over the course of 25 washes is operable to provide satisfactory results for dye systems that present linear or polynomial wash fastness. In one example, a change every 5 wash cycles of about 5 points is used such that the color change is perceivable to the human eye for dye systems that present linear wash fastness. If the wash fastness curve is polynomial, the curve is operable to plateau at a certain wash cycle value with no further change. However, a difference is perceivable to the end-user leading up to the point of plateau. In one example, the presently disclosed dye systems provide linear rates of change in ΔE versus number of wash cycles, polynomial rates of change in ΔE versus number of wash cycles, or combinations thereof. The dye systems of the present disclosure provide qualitative or quantitative wash cycle indicators providing end-user indication of a number of wash cycles including, but not limited to, 5, 15, 20, and/or 25 wash cycles.

In one example, a single dye was used in the dye system with dye add-on %, prepared as a dye system indicator for use with fabric or textiles. Add-on % was done by determining the weight of the un-dyed fabric and taking 1% of that weight to as the add-on amount needed. For example, fabric samples were about 10 grams, so to get the amount of dye added to the bath, a calculation was performed of 1% of 10 grams fabric to provide 0.10 grams of dye, which was added to the bath. Dyes were not added together in the same bath, rather the fabric was dyed in two different steps using separate dye baths. Thus, for example, combinations that were 1% dye A and 1% dye B, with the total add-on % being 2%, were achieved with separate dye baths at a concentration that was 1% of each dye. In one example, a plurality of different percent add-ons for a single dye, were used for a predetermined number of wash cycles. Thus, as shown in FIG. 14, Basic Blue 3 with 0.5% Basic Blue 3 add-on (140); 1% Basic Blue 3 add-on (142); and 2% Basic Blue 3 (144) was used for a predetermined wash cycle indicator, i.e., 20 wash cycles. As shown in FIG. 14, the regression of ΔE₀₀ values vs. wash cycles for the different percent add-ons for Basic Blue 3, determined at 5 wash cycle individuals up to a total of 20 wash cycles is shown, and provides a functioning predetermined wash cycle indicator for fabric or textiles.

In one example, combinations of one or more dyes are used, where one dye has high wash fastness (e.g., permanent dye, ΔE₀₀<10) and another dye has low wash fastness (e.g., leaving dye, ΔE₀₀>20), using only 1% add-ons of each dye.

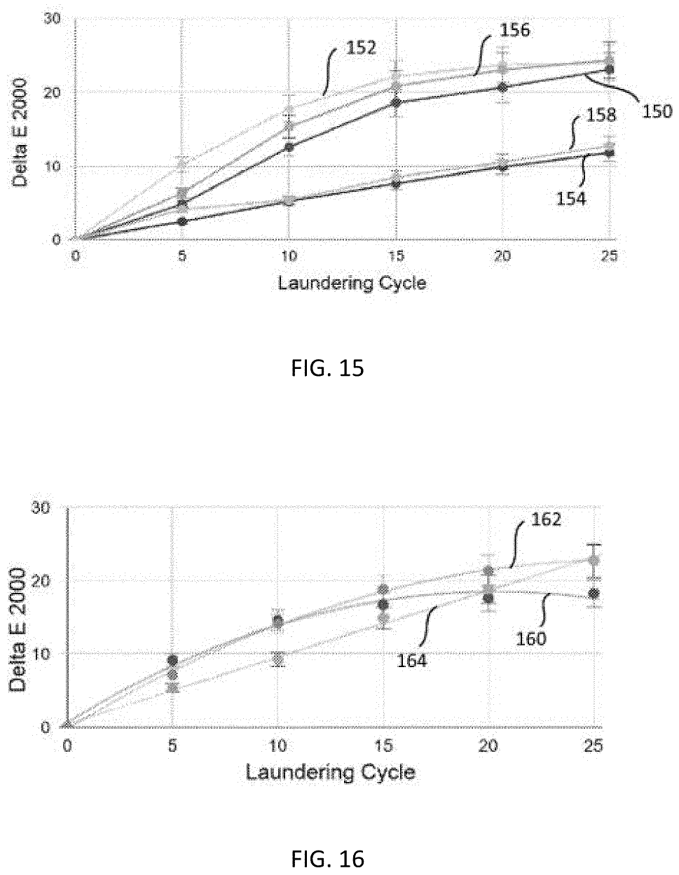

In one example, a dual dye, two color dye system was used. Determination of color combinations was determined based on whether or not the dyes were permanent (e.g., high color fastness/ΔE<10) or transient (e.g., low color fastness/ΔE>20) and by ensuring that color combinations result in visual changes, for example, but not limitation, such as blue/yellow/green, and red/blue/purple combinations. A combination of blue and yellow dyes, or blue and red or purple dyes, are used and ΔE₂₀₀₀ vs. wash cycle was determined. The data in FIG. 14 shows that the 2-color dye systems of Blue 3/Direct Yellow 12 (140); Basic Blue 3/Acid Yellow 17 with formic acid (142); and Basic Blue 3/Mordant Yellow 3 (146) provided ΔE₂₀₀₀ values greater than 20 over 25 wash cycles, beginning at 15 wash cycles and thus provided wash cycle indicators for fabrics and textiles. The dual dye, 2-color dye systems shown in FIG. 15 include Blue 3/Direct Yellow 12 (150); Basic Blue 3/Acid Yellow 17 with formic acid (152); Acid Blue 45/Basic Violet 10 (154); Basic Blue 3/Mordant Yellow 3 (156); and Disperse Red 167/Direct Blue 86 (158).

In one example, a three-component dye system was used. As shown in FIG. 16, an exemplary dye system is shown including three different combinations of one or more indicator dyes, each combination configured to provide visual indication for a predetermined number of washes. For example, an exemplary 3-dye system includes 0.5% Direct Blue 86 and 0.2% Basic Yellow 2 (160); 0.5% Basic Blue 3 (162); and 1% Basic Blue 3 and 2% Direct Yellow 12 (164), where the group of Direct Blue 86 and 0.2% Basic Yellow 2 was used for indicating a visual change after approximately 15 washes, where 0.5% Basic Blue 3 alone was used for indicating a visual change of approximately 20 washes; and the group of 1% Basic Blue 3 and 2% Direct Yellow 12 was used for indicating a visual change of approximately 25 washes. Thus, collectively, the three-component dye system provided a visual indication suitable for use on a fabric or garment to indicate a wash cycle history of approximately 15 washes, approximately 20 washes, and approximately 25 washes.

The 1% Basic Blue 3 and 2% Direct Yellow 12 (used for indicating approximately 25 washes) and the 0.5% Basic Blue 3 (used for indicating approximately 20 washes) had a ΔE₀₀ values of 22.67 and 22.77, respectively. In comparison, 0.5% Direct Blue 86 and 0.2% Basic Yellow 2 after 15 washes had a ΔE₀₀ of 18.28. In one example, the one or more dyes of the dye system configured for providing visual indication of the maximum number of wash cycles, for example 25 or more washes, had a substantially linear regression of ΔE₂₀₀₀ value vs. wash cycles.

Methods of Indicator Production

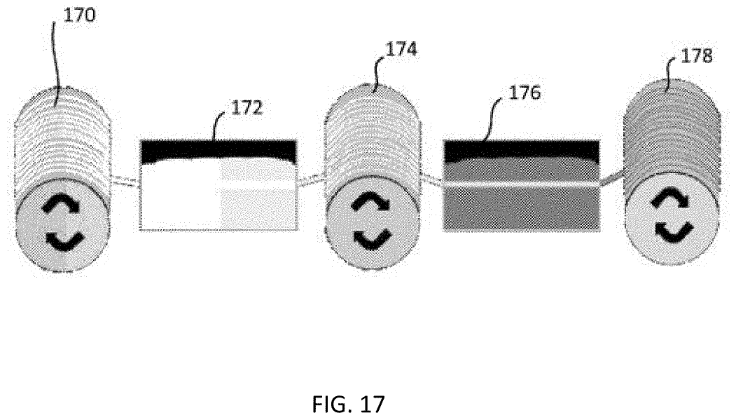

In one example, a continuous, semi-continuous, or batch yarn dyeing process is operable to be implemented. In one example, a continuous dyeing process, as shown in FIG. 17, where feed roll 170 of yarn is introduced to first dye bath 172 and collected by take-up roll 174 and subsequently introduced to second dye bath 176 and collected on spool 178. In one example, dwell time in each of the dye baths is 30 minutes at a temperature of 100° C. Not depicted in FIG. 17 is the intermediary rinse and drying phase of the dyed yarns going from one dye bath to the other. A similar set-up is operable to be used to continuously dye fabric through three or more dye baths.

In one example, a known initial dye bath concentration is operable to be monitored using conventional techniques for a given period of time, and a dynamic concentration of the dye calculated. In one embodiment, the dynamic concentration is operable to be adjusted automatically to ensure that the same percent add-on is present in the dye bath. In one example, color differences are operable to be determined using the yarn rather than the fabric and correlated to the visual change analysis for the fabric.

Thus, in one example a dye system including various two-color combinations were washed and data was collected over the 25 wash cycles providing a functional wash cycle sensor for 15, 20, and 25 wash cycles. In another example, a percent add-on of dyes was determined to determine the rate of color change over the course of 25 washed and a best fit analysis was used to provide a 15, 20, and 25 wash cycle sensor. Thus, for example, an exemplary 2 color combination dye system was developed including 0.5% Direct Blue 86 and 0.2% Basic Yellow 2 for providing a visual indication of change after a 15 wash cycle, 0.5% Basic Blue 3 for providing a visual indication of change after a 20 wash cycle, and 1% Basic Blue 3 and 2% Direct Yellow 12 for providing a visual indication of change after a 25 wash cycle suitable for use for a fabric or garment.

The present disclosure provides for a continuous dye process that maximizes dye retention/wash fastness and maximizes indicator effectiveness. The present disclosure provides for a dye system compatible with various detergents, wash cycles, and anti-soiling performance of the indicator under various lighting settings.

The present dye system and indicator provides wide process latitude for accommodating wash cycles that are operable to differ in the amount of water used, temperature, and time with acceptable dye release profiles throughout the lifetime of the garment while minimizing leaving dye on the skin during wear. The present dye system and indicator is operable to be incorporated into textile products and/or constructed of natural, synthetic, or blends of yarns, with substantially little, if any, compromise of the product's functionality and comfort. The present dye system and indicator is designed and configured for complimentary reading/indexing instructions that are operable to be presented on product packaging.

Indicator System Including Remote Device

As previously described, in one embodiment, the intermediate or final visual indication presents text or one or more symbols. In one embodiment, the text or one or more symbols includes a code (e.g., quick response (QR) code). In one embodiment, the code (e.g., QR code) is operable to be scanned by a camera incorporated into a remote device (e.g., smartphone, tablet, computer). In one embodiment, the code (e.g., QR code) points to a website or application operable to allow purchase of a garment, article, or textile. In one embodiment, the code (e.g., QR code) provides a coupon for purchase of a garment, article, or textile. In one embodiment, a multi-component system (e.g., two-component, three-component) is used as previously described. In one example, the intermediate state (e.g., 15 washes) reveals a QR code.

In one embodiment, the remote device includes a mobile application. In one embodiment, the mobile application is operable to estimate a number of wash cycles using computer vision. Additional information about image processing and computer vision methods is found in Sonka, M., Hlavac, V., & Boyle, R. (2014). Image processing, analysis, and machine vision. Cengage Learning, which is incorporated herein by reference in its entirety. In one embodiment, the mobile application is operable to estimate a number of wash cycles. In one embodiment, the mobile application is operable to recommend repurchase of a garment, article, or textile based on the estimated number of wash cycles. In one embodiment, the mobile application facilitates the repurchase of the garment, article, or textile.

In one embodiment, the indicator includes a passive radio-frequency identification (RFID) tag. In one embodiment, the passive RFID tag is operable to be read by the remote device. In one embodiment, the passive RFID tag is operable to be scanned prior to each wash cycle. Alternatively, the passive RFID tag is not operable to be scanned until a threshold number of wash cycles is reached (e.g., 15, 20, or 25 wash cycles). Information regarding passive RFID tags is included in WIPO Publication No. WO2020201600, which is incorporated herein by reference in its entirety.

In one embodiment, the passive RFID tag is operable to track a length of use and/or length of time since start date. In one embodiment, the passive RFID tag includes a unique product identification number. For example, and not limitation, a consumer purchases a first garment with a first passive RFID tag and a second garment with a second passive RFID tag. The first passive RFID tag is operable to identify the first garment and the second passive RFID tag is operable to identify the second garment.

Experimental

The conditions of dye bath such as temperature and pH are operable to be varied depending on the uniformity of the fabric and the degree of penetration of dyes in the fibers. The evaluation of dye systems suitable as indicators consisted of a CIE color difference test after washing in the standard washing machine and laundrometer. The visual change of the indicator was determined using AATCC EP7-EP7-2015: “Instrumental Assessment of the Change in Color of a Test Specimen,” which is incorporated herein by reference in its entirety. In this procedure, a spectrophotometer and visual gray scale assessments were performed before and after treatment (wash cycle) to the fabric. A ΔE value is obtained for each specimen before and after each subsequent wash cycle. ΔE is calculated by using Equation (I):

ΔE=(ΔL²+Δa²+Δb²)^{1/2} Equation (I)

where ΔL is the difference in lightness from black to white, Δa is the difference in color from red to green, and Δb is the difference in color from blue to yellow.

A threshold ΔE₀₀ value of about 20-50 was used so as to provide a range of acceptable values that correlate to a detectable visual difference to the human eye. Values below 20 will likely have an unperceivable difference to the human eye or not be different in the case where the ΔE₀₀ value is initially 0. The ΔE₀₀ values were used to analyze the rates of color depth change for different percent add-on per fabric weight samples.

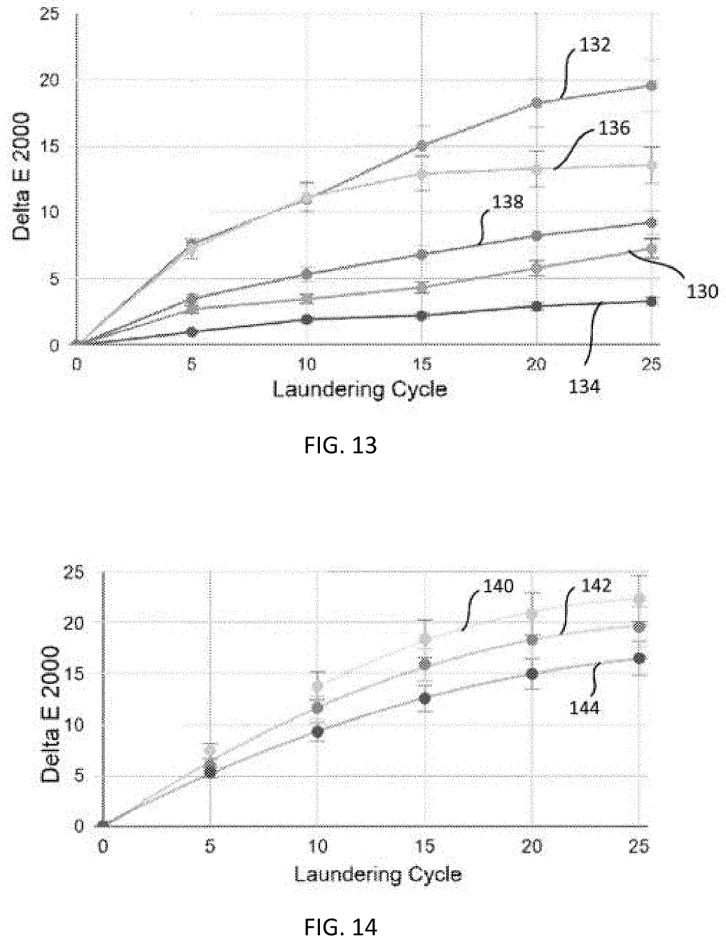

A stock solution using water and dye was used to create dye baths in Ahiba-Labomat beakers with a maximum volume of 200 milliliters, in which samples were introduced. The Ahiba-Labomat was set to have the maximum ramp rate, to have a dwell time of 30 minutes, and to maintain a temperature of 100° C. After dyeing, the samples were rinsed and dried by first running the fabric through a padder and then laying the fabrics out to dry overnight in the open air. After drying, the samples underwent color assessment using a Color i7 Benchtop Spectrophotometer with the following settings: specular included, UV excluded, observer angle of 10°, illuminant D65, an average of 4 readings per sample, and medium to large aperture to obtain initial ΔE₀₀ values. All of the fabric samples were dyed using the same conditions and at a temperature of 100° C. for 30 minutes. After dyeing, the samples were washed 25 times to evaluate the degradation/color fastness of each exemplary dye, an example ΔE₀₀ versus wash cycles is shown in FIG. 13 for Basic Violet 10 (130), Basic Blue 3 (132), Basic Violet 3 (134), Basic Yellow 2 (136), and Basic Red 18 (138).

Once the fabric samples were dyed, dried, and the initial ΔE₀₀ values obtained, a detergent solution of 0.37% AATCC detergent and warm water was prepared. One of the fabric samples, 200 milliliters of detergent solution, and 10 metal ball bearings were added to the launderometer beakers, which were subsequently sealed and then placed in the launderometer to run for 45 minutes at a temperature of 40° C. After a cycle of the launderometer finished, the fabric was, rinsed, padded, and placed in a drying oven for 20 minutes before measuring color change. Each fabric sample underwent 5 launderometer cycles which is equivalent to 25 traditional washing cycles and color was assessed after each cycle resulting in 6 different color readings, including the initial color assessment before laundering.

Wash cycle testing was performed in accordance with AATCC 61: “Test for Colorfastness to Laundering—Accelerated procedure,” which is incorporated herein by reference in its entirety. For the washing procedure, the recommended washing instructions commencement for fabrics and garments containing medicament were used as wash cycle parameters, e.g., 0.37% AATCC laundering detergent, 10 ball bearings, temperature of 40° C., and a wash cycle of 45 minutes in a launderometer.

In one example, test yarns were prepared and varying concentration solutions of extracted yarn including therapeutic agents (e.g., capsaicin) were prepared. Combinations of Gibbs reagent solution with yarn extract solution resulted in intense bright blue solutions for such reactions. The intensity of blue varied with concentration as expected.

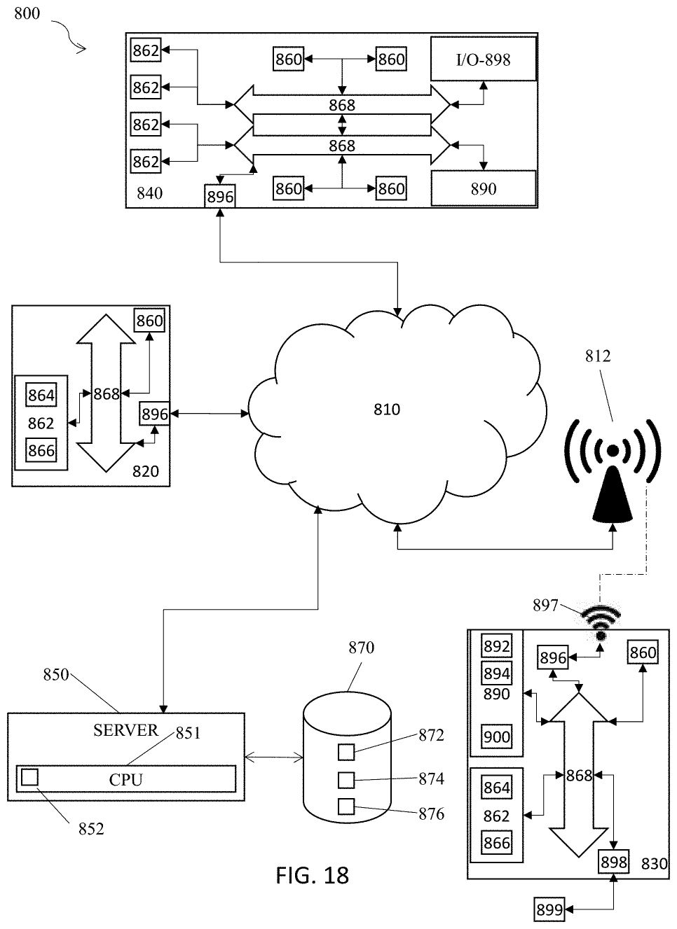

FIG. 18 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, signal generation devices (e.g., speakers), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 18, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 18, is operable to include other components that are not explicitly shown in FIG. 18, or is operable to utilize an architecture completely different than that shown in FIG. 18. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A wearable article comprising:
a yarn, a fiber, or a section of fabric;
at least one active composition applied to the yarn, the fiber, or the section of fabric; and
an indicator;
wherein the yarn, the fiber, or the section of fabric releases the at least one active composition when in contact with a skin surface of a wearer; and
wherein the indicator provides a visual change that corresponds to an amount of the at least one active composition in the yarn, the fiber, or the section of fabric.

2. The wearable article of claim 1, wherein the indicator undergoes a chemical reaction to produce the visual change.

3. The wearable article of claim 1, wherein the wearable article has an initial state, an intermediate state, and a final state, wherein the initial state does not provide a contrasting indication, wherein the intermediate state provides the contrasting indication, wherein the final state provides a maximum contrasting indication, and wherein the maximum contrasting indication provides a constant contrasting indication.

4. The wearable article of claim 1, wherein the visual change includes a quick response (QR) code, wherein, upon being scanned by a camera or sensor of an electronic device, the QR code provides a link to a website or opens an application to facilitate purchase of the wearable article.

5. The wearable article of claim 1, wherein the indicator is a colorimetric indicator.

6. The wearable article of claim 1, wherein the indicator is a quinone or a para-substituted phenolic compound.

7. The wearable article of claim 1, wherein the at least one active composition has a first release rate from the yarn, the fiber, or the section of fabric when in contact with the skin surface of the wearer, wherein the indicator has a second release rate from the yarn, the fiber, or the section of fabric, and wherein the second release rate is substantially similar to the first release rate to correlate with an amount of the at least one active composition of the yarn, the fiber, or the section of fabric.

8. The wearable article of claim 1, wherein the indicator is associated with, chemically bonded to, or complexed with the at least one active composition.

9. The wearable article of claim 1, wherein the at least one active composition includes a capsaicinoid.

10. The wearable article of claim 1, wherein the wearable article is an ankle sleeve, an arm sleeve, a calf sleeve, a knee sleeve, a lower leg sleeve, a wrist sleeve, a wrap, a sock, an insole, a glove, tights, leggings, partial leggings, pants, partial pants, a shirt, or a partial shirt.

11. A wearable article with an indicator comprising:
a yarn, a fiber, or a section of fabric comprising:
visual indicia that correspond to an amount of at least one active composition applied to the yarn, the fiber, or the section of fabric;
wherein the yarn, the fiber, or the section of fabric releases the at least one active composition when in contact with a skin surface of a wearer;
wherein the visual indicia undergo a visual change that corresponds to the amount of the at least one active composition present in the yarn, the fiber, or the section of fabric after one or more uses or wash cycles of the yarn, the fiber, or the section of fabric; and
wherein the visual change results from a chemical reaction.

12. The wearable article of claim 11, wherein the chemical reaction produces or reduces a visual presence of a color, a symbol, and/or text characters.

13. The wearable article of claim 11, wherein the chemical reaction comprises the at least one active composition and one or more reactants.

14. The wearable article of claim 13, wherein the chemical reaction comprises the one or more reactants and a second reactant.

15. The wearable article of claim 13, wherein the one or more reactants are coupled to or immobilized on an inorganic support or an organic support.

16. The wearable article of claim 13, wherein the one or more reactants comprises a quinone or a para-substituted phenolic compound.

17. The wearable article of claim 13, wherein the one or more reactants includes Gibbs reagent.

18. The wearable article of claim 11, wherein the chemical reaction is reversible.

19. The wearable article of claim 11, wherein the chemical reaction is an acid-base reaction, a decomposition reaction, or an oxidation-reduction reaction.

20. A wearable article comprising:
a yarn, a fiber, or a section of fabric having a first color associated therewith;
at least one active composition applied to the yarn, the fiber, or the section of fabric; and
an indicator associated with, chemically bonded to, or complexed with the at least one active composition;
wherein the yarn, the fiber, or the section of fabric releases the at least one active composition when in contact with a skin surface of a wearer;
wherein the indicator has a second color associated therewith; and
wherein an amount of the at least one active composition is correlated with the second color of the indicator.
